(19) United States
(12) Patent Application Publication
(10) Pub. No.: US 2012/0214205 A1
(43) Pub. Date: Aug. 23, 2012

(54) PLANT BIOMASS PRETREATMENT METHOD

(75) Inventors: Kazuhiro Iida, Miyoshi-shi (JP); Kazuhide Tabata, Miyoshi-shi (JP); Takashi Nagase, Nisshin-shi (JP); Sadao Ikeda, Toyota-shi (JP); Kenji Yamada, Nagoya-shi (JP)

(73) Assignee: TOYOTA JIDOSHA KABUSHIKI KAISHA, Toyota-shi, Aichi (JP)

(21) Appl. No.: 13/391,184

(22) PCT Filed: Aug. 18, 2009
(86) PCT No.: PCT/JP2009/064441

§ 371 Date: May 7, 2012

(51) Int. Cl. C12P 19/00

(52) U.S. Cl. 435/72

(57) ABSTRACT

A plant biomass pretreatment method which allows prompt pretreatment of plant biomass with simple equipment is provided. The method includes continuously performing in sequence, inside an extruder, pretreatment steps of coarsely crushing the plant biomass to a predefined size or smaller, adding a decomposing agent(s) to the coarsely crushed plant biomass, applying a hot compressed water treatment(s) to the plant biomass with the decomposing agent added thereto, and performing saccharification preparation for mixing the plant biomass with the hot compressed water treatment applied thereto with an enzyme(s) for saccharifying the plant biomass.

### TECHNICAL FIELD

The present invention relates to a plant biomass pretreatment method for producing ethanol from plant biomass through enzymatic decomposing.

### BACKGROUND ART

Various methods have conventionally been proposed for producing saccharides from plant biomass and fermenting the generated saccharides to produce ethanol.

Patent Document 1 discloses a technique of a pretreatment method for conveying wood-based biomass while agitating and mixing the biomass with a screw inside an extruder, warming the wood-based biomass with steam so as to swell the biomass in the process of conveyance, and introducing the swelling-processed wood-based biomass into acid treatment equipment for application of an acid treatment. However, the acid treatment has issues of waste treatment and environmental loads.

Accordingly, an enzymatic method has been proposed in which cellulose and hemicellulose contained in biomass are degraded by enzymes into saccharides and the saccharides are then fermented to produce ethanol.

Since cellulose and hemicellulose in plant cells exist in the form of being protected by lignin, it is necessary to break down the lignin such that the cellulose and the hemicellulose are exposed to be degraded by enzymes. Since cellulose and hemicellulose have strong binding force, it is also necessary to slightly degrade in advance the structures of the cellulose and the hemicellulose in order that the bonds thereof are degraded by enzymes. Such lignin breakdown treatment and structural decomposing treatment of cellulose and hemicellulose are referred to as a pretreatment.

As a pretreatment, such methods have been devised including decomposing with dilute sulfuric acid, steam explosion, ammonia fiber explosion, methods using hot water and supercritical water, microbial decomposing, fine crushing, and chemicals treatment. Patent Document 1 discloses a method for breaking down lignin by using an extruder to shear wood chips under heat and pressure and to extrude the wood chips to the atmosphere such that the wood chips are swelled.
Patent Document 1: JP Patent Publication (Kokai) No. 2007-202518 A

### DISCLOSURE OF THE INVENTION

### Problems to be Solved by the Invention

The technique described in Patent Document 1 is to break down lignin to expose cellulose, which makes it necessary to separately perform a step of structural decomposing of cellulose and hemicellulose and a saccharification preparation step of mixing enzymes with materials to be treated. Therefore, the technique was inefficient, took time and energy, and was also costly due to the cost of equipment therefor and the like.

In view of the above-stated problem, an object of the present invention is to provide a plant biomass pretreatment method which allows prompt pretreatment of plant biomass with simple equipment.

### Means for Solving the Problems

In order to accomplish the above object, a plant biomass pretreatment method according to the present invention is a plant biomass pretreatment method for performing a pretreatment to produce ethanol from the plant biomass with use of an enzyme(s), the method including continuously performing in sequence, inside an extruder, pretreatment steps of: coarsely crushing the plant biomass to a predefined size or smaller; adding a decomposing agent(s) to the coarsely crushed plant biomass; applying a hot compressed water treatment(s) to the plant biomass with the decomposing agent added thereto; and performing saccharification preparation for mixing the plant biomass with the hot compressed water treatment applied thereto with an enzyme(s) for saccharifying the plant biomass (claim 1).

According to the pretreatment method of the plant biomass in the present invention, the following pretreatment steps are continuously performed in sequence inside an extruder: coarsely crushing the plant biomass to a predefined size or smaller, adding a decomposing agent(s) for applying a hot compressed water treatment(s), and performing saccharification preparation for mixing the plant biomass with enzymes. Consequently, each of the coarse crushing treatment, the hot compressed water treatment, and the saccharification preparation, which were conventionally performed in a separate and independent manner, can be performed consistently. Therefore, an efficient pretreatment can be performed, the cost of equipment can be reduced due to simplified equipment, and thereby lower costs can be achieved.

According to the plant biomass pretreatment method in the present invention, the extruder preferably includes: a cylinder having a passage which includes a feed port formed for feeding the plant biomass in one end and a discharge port formed for discharging a material(s) to be pretreated in the other end; and a screw line(s) which is arranged inside the passage of the cylinder and which includes a delivery section(s) for delivering the plant biomass toward the discharge port, a kneading section(s) for kneading the plant biomass, and a resistance element(s) for providing delivering resistance to the plant biomass, the extruder having in sequence from an upstream side to a downstream side in the passage of the cylinder: a coarse crushing zone(s) for coarsely crushing the plant biomass to a predefined size or smaller; a hot compressed water treatment zone(s) for applying a hot compressed water treatment(s) to the plant biomass coarsely crushed in the coarse crushing zone; a cooling zone(s) for cooling the plant biomass with the hot compressed water treatment applied thereto in the hot compressed water treatment zone; a saccharification preparation zone(s) for mixing the plant biomass cooled in the cooling zone with an enzyme(s); and a discharge zone(s) for discharging the plant biomass mixed with the enzyme in the saccharification preparation zone as a material(s) to be pretreated (claim 2).

According to the plant biomass pretreatment method in the present invention, it is preferable that a screw line having at least one or more types of screw segments, including a special gear kneader(s) or a special fluffer ring(s), is placed in a plant biomass high filling zone(s) formed by the resistance element of the screw line on an upstream side of the resistance element (claim 3).

According to the plant biomass pretreatment method in the present invention, it is preferable that a screw line(s) having at least one or more types of screw segments, including a forward kneading disk(s), a backward kneading disk(s), an perpendicular kneading disk(s), a special gear kneader(s), and a special fluffer ring(s), is placed in the coarse crushing zone (claim 4).

According to the plant biomass pretreatment method in the present invention, it is preferable that a screw line(s) having at least one or more types of screw segments, including a reverse full flight(s), a special gear kneader(s), or a special fluffer ring(s), is placed in the hot compressed water treatment zone, a resistance element(s) having a special seal ring(s) is placed respectively in an upstream end and in a downstream end of the hot compressed water treatment zone, and the plant biomass is sheared and kneaded under heat and pressure in the hot compressed water treatment zone (claim 5).

According to the plant biomass pretreatment method in the present invention, it is preferable that the resistance elements placed in the hot compressed water treatment zone are set such that the resistance element on a downstream side is higher in resistance than the resistance element on an upstream side (claim 6).

According to the plant biomass pretreatment method in the present invention, it is preferable that a decomposing agent feed part(s) for feeding a decomposing agent(s) to the hot compressed water treatment zone in the passage, a coolant feed part(s) for feeding a coolant(s) to the cooling zone(s), and an enzyme feed part(s) for feeding an enzyme(s) to the saccharification preparation zone are each provided (claim 7).

According to the plant biomass pretreatment method in the present invention, it is preferable that a plurality of the decomposing agent feed parts are provided at predetermined intervals along the passage of the cylinder, and a feed amount of the decomposing agent is set to be higher on the upstream side than on the downstream side (claim 8).

According to the plant biomass pretreatment method in the present invention, the feed amount of the decomposing agent is preferably set at 5 to 150 weight parts with respect to 100 weight parts of the plant biomass (claim 9).

According to the plant biomass pretreatment method in the present invention, it is preferable that heat and pressure are applied to the extruder with a pressure inside the cylinder being 1 to 30 MPa and a temperature of the hot compressed water treatment zone being 130° C. to 350° C. (claim 10).

According to the plant biomass pretreatment method in the present invention, it is preferable that a screw line(s) having at least one or more types of screw segments, including a forward kneading disk(s), a backward kneading disk(s), and an perpendicular kneading disk(s), is placed in the discharge zone (claim 11).

According to the plant biomass pretreatment method in the present invention, it is preferable that the cylinder has a vent(s) in the discharge zone for discharging gas inside the passage, and the gas inside the cylinder is discharged through the vent (claim 12).

### Advantages of the Invention

According to the plant biomass pretreatment method in the present invention, the following pretreatment steps are continuously performed in sequence inside an extruder: coarsely crushing the plant biomass to a predefined size or smaller, applying a hot compressed water treatment by adding a decomposing agent(s) and crushing the plant biomass, and performing saccharification preparation for mixing the plant biomass with enzymes. Consequently, each of the coarse crushing treatment, the hot compressed water treatment, and the saccharification preparation, which were conventionally performed in a separate and independent manner, can be performed consistently. Therefore, an efficient pretreatment can be performed, the cost of equipment can be reduced due to simplified equipment, and thereby lower costs can be achieved.

### BRIEF DESCRIPTION OF THE DRAWINGS

FIG. 1 is a flow chart for explaining a pretreatment method of plant biomass feedstock;

FIG. 2 is a schematic view showing configurations of a cylinder and a screw line of a screw extruder;

FIG. 3 is a view showing a configuration of a forward full flight;

FIG. 4 is a view showing a configuration of a reverse full flight;

FIG. 5 is a view showing a configuration of a forward double-threaded screw kneading disk;

FIG. 6 is a view showing a configuration of a backward double-threaded screw kneading disk;

FIG. 7 is a view showing a configuration of an perpendicular double-threaded screw kneading disk;

FIG. 8 is a view showing a configuration of a special gear kneader;

FIG. 9 is a view of FIG. 8 viewed from an arrow U1 direction;

FIG. 10 is a schematic cross sectional view showing a gear fitting state of the special gear kneader in FIG. 8;

FIG. 11 is a partially enlarged view showing a tooth section shown in FIG. 9;

FIG. 12 is a view showing another example of a special gear kneader;

FIG. 13 is a view of FIG. 12 viewed from an arrow U1 direction;

FIG. 14 is a schematic cross sectional view showing a gear fitting state of the special gear kneader in FIG. 12;

FIG. 15 is a partially enlarged view showing a tooth section shown in FIG. 13;

FIG. 16 is a view showing another example of a special gear kneader;

FIG. 17 is a view of FIG. 16 viewed from an arrow U1 direction;

FIG. 18 is a schematic cross sectional view showing a gear fitting state of the special gear kneader in FIG. 16;

FIG. 19 is a partially enlarged view showing a tooth section shown in FIG. 17;

FIG. 20 is a view showing an example of a special fluffer ring;

FIG. 21 is a view of FIG. 20 viewed from an arrow U1 direction;

FIG. 22 is a view showing an example of a seal ring;

FIG. 23 is a view of FIG. 22 viewed from an arrow U1 direction;

FIG. 24 is a cross sectional view of FIG. 23 taken along line A-A;

FIG. 25 is a view showing another example of a seal ring;

FIG. 26 is a view of FIG. 25 viewed from an arrow U1 direction;

FIG. 27 is a cross sectional view of FIG. 26 taken along line B-B;

FIG. 28 is a view showing another example of a seal ring;

FIG. 29 is a view of FIG. 28 viewed from an arrow U1 direction;

FIG. 30 is a cross sectional view of FIG. 29 taken along line C-C;

FIG. 31 is an enlarged view showing a principal part of FIG. 28;

FIG. 32 is a view showing a lead groove provided on a seal ring in cross section;

FIG. 33 is a view showing a lead groove provided on a seal ring in cross section;

FIG. 34 is a view showing a lead groove provided on a seal ring in cross section;

FIG. 35 is a schematic view showing another embodiment of a twin screw extruder of the present invention;

FIG. 36 is a schematic view showing another embodiment of a twin screw extruder of the present invention;

FIG. 37 is a schematic view showing another embodiment of a twin screw extruder of the present invention;

FIG. 38 is a schematic view of a gear kneader included in a conventional twin screw extruder; and

FIG. 39 is an enlarged view showing a principal part of FIG. 38.

### DESCRIPTION OF SYMBOLS

1 . . . cylinder, 1a . . . passage, 2 . . . feed port, 3 . . . discharge port, 4 . . . decomposing agent feed part, 4a . . . first feed part, 4b . . . second feed part, 5 . . . coolant feed part, 6 . . . enzyme feed part, 11 . . . coarse crushing zone, 12 . . . hot compressed water treatment zone, 12A . . . upstream zone, 12B . . . downstream zone, 13 . . . cooling zone, 14 . . . saccharification preparation zone, 15 . . . discharge zone, 21-25 . . . screw line, 31-35 . . . resistance element, 50 . . . forward full flight, 52 . . . reverse full flight, 43 . . . forward double-threaded screw kneading disk, 54 . . . backward double-threaded screw kneading disk, 45 . . . perpendicular double-threaded screw kneading disk, 100 . . . special gear kneader, 200 . . . special fluffer ring, 300 . . . special seal ring

### BEST MODE FOR CARRYING OUT THE INVENTION

Embodiments of the present invention will be described hereinbelow with reference to accompanying drawings.

### EMBODIMENT

### Embodiment 1

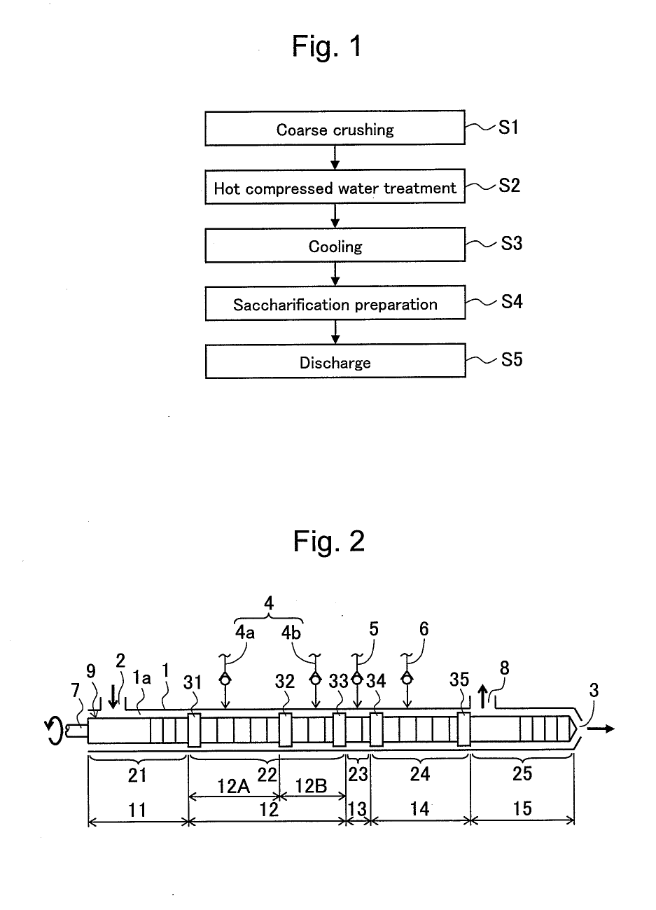

FIG. 1 is a flow chart for explaining a pretreatment method of plant biomass feedstock in the present invention, and FIG. 2 is a schematic view showing configurations of a cylinder and a screw line of a screw extruder for use in the pretreatment method.

The pretreatment method of plant biomass feedstock in the present invention includes, as shown in FIG. 1, a coarse crushing step S1, a hot compressed water treatment step S2, a cooling step S3, a saccharification preparation step S4, and a discharging step S5, and these respective steps are continuously performed in sequence inside a cylinder 1 of a screw extruder shown in FIG. 2.

Used as a screw extruder is a coaxial-rotating twin screw extruder having two screw lines which are parallely placed and rotate in the same direction, the extruder including a cylinder 1 having a linearly extending passage 1a.

The cylinder 1 has a feed port 2 formed for feeding plant biomass (nonliquid materials) such as wood chips in one end portion of the passage 1a and a discharge port 3 formed for discharging pretreated plant biomass feedstock in the passage 1a in the other end portion of the passage 1a.

Inside the passage 1a of the cylinder 1, two screw shafts 7 in a pair connected to an unshown drive motor are arranged in parallel. A screw line 9 is configured by attaching various screw segments, including full flights 50, 52 and kneading disks 54, 56, 58, to a pair of these screw shafts 7 in series in appropriate combination.

The screw line 9 constitutes a delivery means including a plurality of delivery sections which integrally rotate with rotation of the screw shaft 7 by the drive motor in the passage 1a and which deliver materials to be treated toward the discharge port 3 with the rotation, a kneading/shearing section for shearing and kneading materials to be treated, and resistance elements for providing delivering resistance to the materials to be treated.

Inside the passage 1a of the cylinder 1, a coarse crushing zone 11, a hot compressed water treatment zone 12, a cooling zone 13, a saccharification preparation zone 14, and a discharge zone 15 are configured in series. The hot compressed water treatment zone 12 is formed between resistance elements 31 and 33, which are separately provided on the upstream side and the downstream side in a delivery direction along the passage 1a. In this embodiment, resistance elements 31, 32, 33 are provided respectively in an upstream section, an intermediate section and a downstream section of the hot compressed water treatment zone 12, by which an upstream zone 12A and a downstream zone 12B are formed.

In the cylinder 1, there are provided a decomposing agent feed part(s) 4 for feeding a decomposing agent(s) to the hot compressed water treatment zone 12, a coolant feed part(s) 5 for feeding a coolant(s) to the cooling zone 13, and an enzyme feed part(s) 6 for feeding enzymes to the saccharification preparation zone 14.

A plurality of decomposing agent feed parts 4 are provided at predetermined intervals in a longitudinal direction of the passage 1a, and in this embodiment, a first feed part 4a is provided in the upstream zone 12A, while a second feed part 4b is provided in the downstream zone 12B. A feed amount of the decomposing agent per unit time is set to have a relationship of (first feed part 4a>second feed part 4b). Examples of the decomposing agent to be used include water such as cold water and hot water, acids, alkalis, solvents, decay fungi, and supercritical liquids, and the agent is fed into the passage 1a from the decomposing agent feed part 4 and is added to plant biomass feedstock.

It is to be noted that the decomposing agent feed part 4 may be provided in the coarse crushing zone 11 to feed the decomposing agent to the coarse crushing zone 11. Decomposing agents such as acids and decay fungi for example are fed to the coarse crushing zone 11, and thereby crushing of plant biomass feedstock and adding of the decomposing agents can be simultaneously performed, and higher efficiency can be achieved.

The coolant feed part 5 feeds a coolant(s) such as liquid nitrogen to the cooling zone 13 to cool the plant biomass feedstock which were heated to high temperature in the hot compressed water treatment zone 12, such that the temperature of the plant biomass feedstock is adjusted to the temperature optimal for the activity of enzymes. The enzyme feed part 6 feeds enzymes to plant biomass feedstock. The enzymes are mixed with the plant biomass feedstock in the saccharification preparation zone. A plurality of the coolant feed parts 5 and the enzyme feed parts 6 may each be provided at predetermined intervals in the longitudinal direction inside the passage 1a.

The cylinder 1 is provided with an unshown heating heater, which can heat the plant biomass feedstock in the hot compressed water treatment zone 12 and maintain the plant biomass feedstock in a high-temperature state. An appropriate amount of the plant biomass is fed into the passage 1a through the feed port 2 at the right time. In this embodiment, wood-based biomass such as wood chips is used.

The steps S1 to S5 will be described hereinbelow in detail.

In the coarse crushing step S1, chip-like plant biomass feedstock are mechanically crushed into coarsely crushed objects of a predefined size or smaller by shearing, friction, dispersion, diffusion, and kneading by rotation of the screw line 9. The plant biomass feedstock as the coarsely crushed objects are delivered from the coarse crushing zone 11 to the hot compressed water treatment zone 12 in the downstream.

A screw line 21 in the coarse crushing zone 11 is composed of an appropriate combination of, for example, the forward full flight 50, the forward double-threaded screw kneading disk 54, the backward double-threaded screw kneading disk 56, and the perpendicular double-threaded screw kneading disk 58. At least one of a special gear kneader(s) 100 and a special fluffer ring(s) 200 is arranged in a high filling zone(s) which is formed within the coarse crushing zone 11 with a high filling rate of the plant biomass feedstock and in a delivery zone(s) for delivering the plant biomass feedstock to the hot compressed water treatment zone 12 in the downstream.

The special gear kneader 100 and the special fluffer ring 200 can generate turbulence in a flow of the plant biomass feedstock in the passage 1a to promote shearing, coarse crushing, kneading, dispersion and decomposing of the plant biomass feedstock. They can also reinforce and stabilize delivery of the plant biomass feedstock to the downstream side and can thereby prevent occurrence of plugs. It is to be noted that the temperature of the plant biomass feedstock in the coarse crushing zone is set at room temperature.

In the hot compressed water treatment step S2, a decomposing agent(s) such as water is fed into the passage 1a from the first feed part 4a and the second feed part 4b and is added to the plant biomass feedstock. Then, a hot compressed water treatment of the plant biomass feedstock is performed by rotation of a screw line 22. In the hot compressed water treatment, the plant biomass feedstock are micronized, kneaded, agitated, dispersed, and degraded with the screw line 22 in hot compressed water.

The screw line 22 in the hot compressed water treatment zone 12 includes the resistance elements 31, 32, 33 for suppressing delivery of the plant biomass feedstock respectively at a most upstream section, a most downstream section, and an intermediate section of the hot compressed water treatment zone 12, and a high filling zone(s) with a high filling rate of the plant biomass feedstock is formed in the upstream side of the resistance elements 31 to 33.

In the hot compressed water treatment zone 12, sealing performance is enhanced by these resistance elements 31 to 33, and the hot compressed water treatment zone 12 is maintained in a high-pressure state where the pressure is equal to or more than the saturated vapor pressure (e.g., 1 to 30 MPa).

The resistance elements 31, 33 include a special seal ring(s) 300, and a space between the special seal ring 300 and an inner wall surface of the cylinder passage 1a is sealed with the plant biomass feedstock to form a sealed state, by which the pressure inside the hot compressed water treatment zone 12 is increased.

In the hot compressed water treatment zone 12, the temperature of the plant biomass feedstock in the hot compressed water treatment zone 12 can be maintained from 130° C. to 350° C. through heating by a heater and with shearing frictional heat by the screw line 9.

Therefore, the hot compressed water treatment zone 12 can be put in a hot compressed water state (high pressure and high temperature), which makes it possible to perform a hydrothermal treatment in which the plant biomass feedstock with a decomposing agent(s) added thereto are swelled and softened. Thus, the hydrothermally-treated plant biomass feedstock can finely be crushed with ease through shearing and kneading with the screw line 22.

In the case where decay fungi are added as decomposing agents, the plant biomass feedstock are maintained from room temperature to 80° C. In the case where supercritical water is added as a decomposing agent, the pressure in the hot compressed water treatment zone 12 is set at a supercritical pressure or higher.

The screw line 22 is composed of an appropriate combination of, for example, the special seal ring 300, the special gear kneader 100, the special fluffer ring 200, the forward full flight 50, the reverse full flight 52, the forward double-threaded screw kneading disk 54, the backward double-threaded screw kneading disk 56, and the perpendicular double-threaded screw kneading disk 58.

The hot compressed water treatment zone 12 is divided into the upstream zone 12A and the downstream zone 12B by the resistance element 32 at the intermediate section. A screw design of the screw line 22 is made such that at least one of the special gear kneader 100 and the special fluffer ring 200 is arranged in each of the high filling zone formed with the resistance elements 31 to 33, a delivery zone(s) for delivering the plant biomass feedstock from the upstream zone 12A to the downstream zone 12B, and a delivery zone(s) for delivering the plant biomass from the downstream zone 12B to the cooling zone 13.

Arranging such a segment as the special gear kneader 100 in the high filling zone makes it possible to achieve prompt micronization, kneading, agitation, dispersion, and decomposing of the plant biomass feedstock, and arranging such a segment as the special gear kneader 100 in the delivery zone makes it possible to prevent compressive force and frictional force from being locally applied to the plant biomass feedstock and to thereby prevent occurrence of plugs.

Each of the resistance elements 31 to 33 of the screw line 22 is composed of a combination of the special seal ring 300, the reverse full flight 32, the special gear kneader 100, and the special fluffer ring 200. The resistance of each of the resistance elements 31 to 33 is set to have a relationship of (resistance element 31 at most upstream section<resistance element 32 at intermediate section<resistance element 33 at most downstream section) such that the resistance is larger toward the downstream side.

Since micronization, kneading, and decomposing of the plant biomass feedstock progress more and their shearing resistance, kneading and diffusion resistance, and flow resistance become smaller toward the downstream side in the hot compressed water treatment zone 12, a clearance between the resistance elements and the inner wall surface of the passage 1a is made smaller toward the downstream side to ensure a proper flow and a filling rate both in the upstream zone 12A and the downstream zone 12B, and thereby diffusibility and dispersibility with the decomposing agent can be maintained and more efficient decomposing can be achieved.

Moreover, since the resistance elements 31 to 33 are placed at the upstream section, the intermediate section, and the downstream section along the flow direction, the plant biomass feedstock are repeatedly subjected to compression and expansion, and thereby efficiency of each treatment can be enhanced.

The first feed part 4a is arranged on the upstream side in the upstream zone 12A, and the second feed part 4b is arranged on the upstream side in the downstream zone 12B. Therefore, a distance for performing the hydrothermal treatment in each zone is set to be as large as possible and the hydrothermal treatment can be performed effectively. In the case where the decomposing agent is water for example, a ratio of the feed amount of the decomposing agent is set at 0.25-3 with respect to the plant biomass feedstock, whereas in the case where the decomposing agents are acids, alkalis, and solvents, the ratio is set at 0.01-1 with respect to the plant biomass feedstock.

Since the hot compressed water treatment zone 12 is held at a high-pressure and high-temperature state with the special seal ring 300, it becomes possible to efficiently perform the hydrothermal treatment which softens the plant biomass feedstock. Therefore, the plant biomass feedstock are finely crushed by shearing, kneading, dispersion and decomposing actions of the screw line 22, and become still finer than the plant biomass feedstock in the coarse crushing zone 11. The first feed part 4a and the second feed part 4b are provided in the same number as for the high filling zones formed inside the hot compressed water treatment zone 12 in order that an effective hydrothermal treatment is performed.

A feed position in the decomposing agent feed part 4 may be set depending on conditions such as pressure and temperature in the hot compressed water treatment zone 12. Feeding a decomposing agent(s) at an appropriate position allows prompt micronization, kneading, agitation, dispersion, and decomposing of the plant biomass feedstock, and makes it possible to prevent feeding of an excessive amount of the treatment agent. The plant biomass feedstock, which were treated in the hot compressed water treatment zone 12, are delivered to the cooling zone 13 positioned in the downstream.

In the cooling step S3, a coolant(s) such as liquid nitrogen is fed into the passage 1a from the coolant feed part 5 to perform a treatment for cooling the plant biomass feedstock in the cooling zone 13. A screw line 23 is composed of a combination of only the screw segments with a delivery function, such as the forward full flight 50.

Since the plant biomass is heated to high temperature in the hot compressed water treatment zone 12, the temperature of the plant biomass immediately after being delivered from the hot compressed water treatment zone 12 is high, and this high temperature is not desirable for enzymes. If enzymes are charged in such a temperature state in the saccharification preparation step S4, saccharification with enzymes may encounter difficulty. Accordingly, the cooling step S3 was provided between the hot compressed water treatment step S12 and the saccharification preparation step S4 to cool the high-temperature plant biomass feedstock to appropriate temperature such that appropriate saccharification with enzymes could be carried out. It is to be noted that the temperature of the plant biomass feedstock in the cooling zone 13 is lowered to 40° C.-50° C. by the coolant.

In the saccharification preparation step S4, a treatment is performed which includes feeding enzymes into the passage 1a from the enzyme feed part 6 and mixing the enzymes with the plant biomass feedstock in the saccharification preparation zone 14.

A screw line 24 in the saccharification preparation zone 14 is composed of an appropriate combination of, for example, the special seal ring 300, the special gear kneader 100, the special fluffer ring 200, the forward full flight 50, the reverse full flight 52, the forward double-threaded screw kneading disk 54, the backward double-threaded screw kneading disk 56, and the perpendicular double-threaded screw kneading disk 58. A predetermined amount of an enzyme liquid is fed into the passage 1a from the enzyme feed part 6 and is added to the plant biomass feedstock within the saccharification preparation zone 14 (e.g., 40 FPU).

The plant biomass feedstock, which have moved as far as to the treatment of the saccharification preparation step S4, gain high viscosity, which may be too high, for example, for operators to carry out through mixing. However, the plant biomass feedstock are mixed by means of the screw line 24 in the saccharification preparation zone 14, and thereby enzymes can sufficiently be mixed into the plant biomass feedstock. Once the plant biomass feedstock are mixed with enzymes in the saccharification preparation zone 14, they are delivered to the discharge zone 15 positioned in the downstream.

In the discharging step S5, a treatment is performed in which the plant biomass with enzymes mixed therein in the saccharification preparation zone 14 is discharged as materials to be pretreated, while at the same time a treatment is performed in which gas components are removed from the plant biomass feedstock with the saccharification preparation subjected thereto. The cylinder 1 is provided with a vent 8 for deaeration. The vent 8, which communicates the discharge zone 15 of the passage 1a with the outside, can discharge a part of gas components in the discharge zone 15.

Discharging a part of gas components through the vent 8 allows proper adjustment of a water content of the decomposing agent in the plant biomass feedstock and also allows removal of unnecessary gas components such that the plant biomass feedstock can be fed in an optimal state to subsequent steps such as the saccharification step. The plant biomass feedstock discharged from the discharge port 3 are converted to ethanol through similar steps to a prior art (saccharification, fermentation, purification).

A screw line 25 in the discharge zone 15 is composed of an appropriate combination of, for example, respective screw segments including the forward double-threaded screw kneading disk 54, the backward double-threaded screw kneading disk 56, and the perpendicular double-threaded screw kneading disk 58. The downstream zone for discharging the plant biomass feedstock from the discharge port 3 is configured such that at least one of the special gear kneader 100 and the special fluffer ring 200 is arranged therein.

According to the above-stated plant biomass pretreatment method in the present invention, the following pretreatment steps are continuously performed in sequence inside an extruder: coarsely crushing the plant biomass to a predefined size or smaller, adding a decomposing agent(s) for applying a hot compressed water treatment, and performing saccharification preparation for mixing the plant biomass with enzymes. Consequently, each of the coarse crushing treatment, the hot compressed water treatment, and the saccharification preparation treatment, which were conventionally performed in a separate and independent manner, can be performed consistently. Therefore, an efficient pretreatment can be performed, the cost of equipment can be reduced due to simplified equipment, and thereby lower costs can be achieved.

### Screw Shape

Hereinbelow, respective screw segments which constitute the screw line 9 in this embodiment will be described.

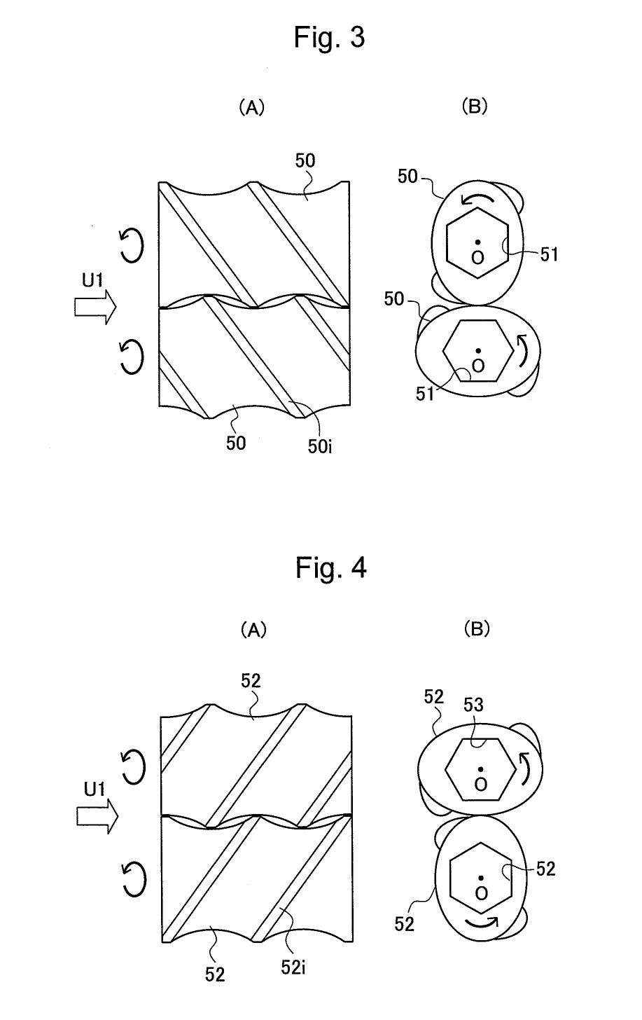

FIGS. 3(A) and 3(B) are views showing an example of a forward full flight, and FIGS. 4(A) and 4(B) are views showing an example of a reverse full flight. In FIG. 3(A) and FIG. 4(B), a generally round-shaped inner wall surface of the passage 1a in the cylinder 1 is omitted.

The forward full flight 50 has a twist orientation shown with a screw line 50i set for ensuring a capability of delivery to the downstream side, while the reverse full flight 52 has a twist orientation shown with a screw line 52i set for reducing the capability of delivery to the downstream side.

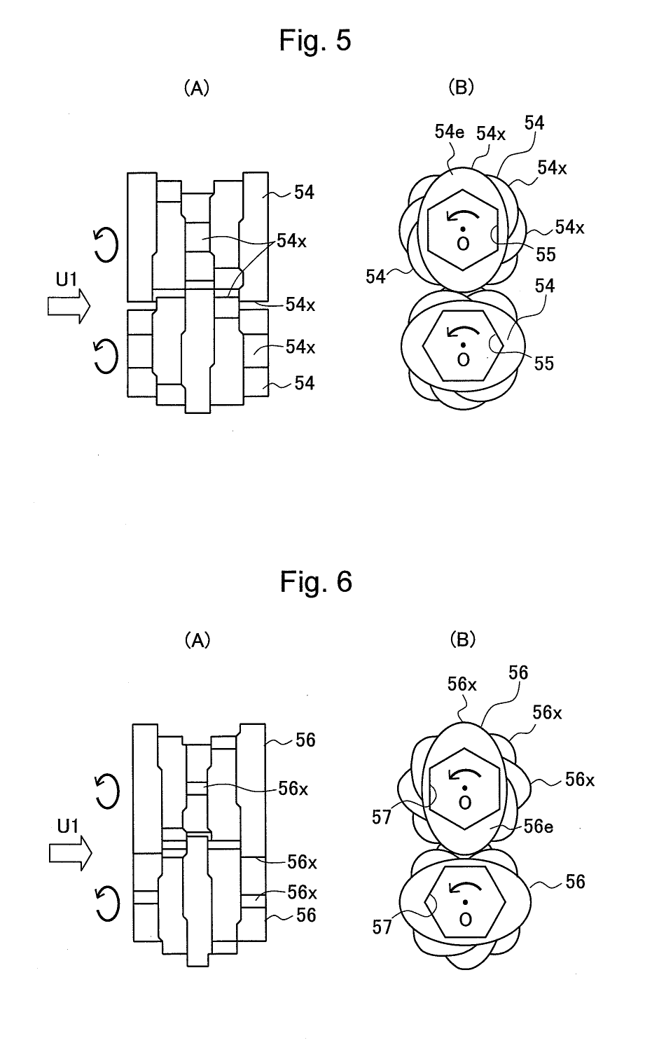

An example of the forward double-threaded screw kneading disk 54 is shown in FIGS. 5(A) and 5(B). The forward double-threaded screw kneading disk 54 is structured to have a generally egg-shaped paddle 54e having top sections 54x, which are arranged in series from top left to bottom right.

An example of the backward double-threaded screw kneading disk 56 is shown in FIGS. 6(A) and 6(B). The backward double-threaded screw kneading disk 56 is structured to have a generally egg-shaped paddle 56e having top sections 56x, which are arranged in series from bottom left to top right.

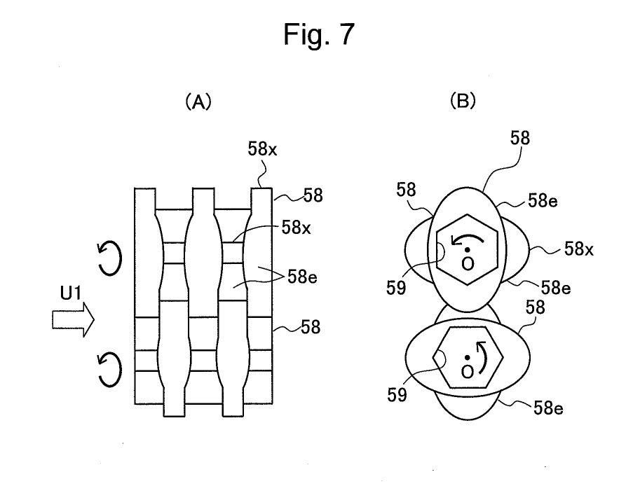

FIGS. 7(A) and 7(B) are views showing an example of the perpendicular double-threaded screw kneading disk 58. The perpendicular double-threaded screw kneading disk 28 is structured to have generally egg-shaped paddles 58e having a top section 58x, the paddles 58e being placed in series at an angle of gradient of 90 degrees. Although the perpendicular double-threaded screw kneading disk 58 has no helical angle and therefore has almost no capability of delivery, it has a high shearing capability and is also high in dispersion and kneading capabilities.

The forward full flight 50, the reverse full flight 52, the forward double-threaded screw kneading disk 54, the backward double-threaded screw kneading disk 56, and the perpendicular double-threaded screw kneading disk 58 have through holes 51, 53, 55, 57, 59 formed along their central axes for receiving and fixing the screw shaft 7 therein.

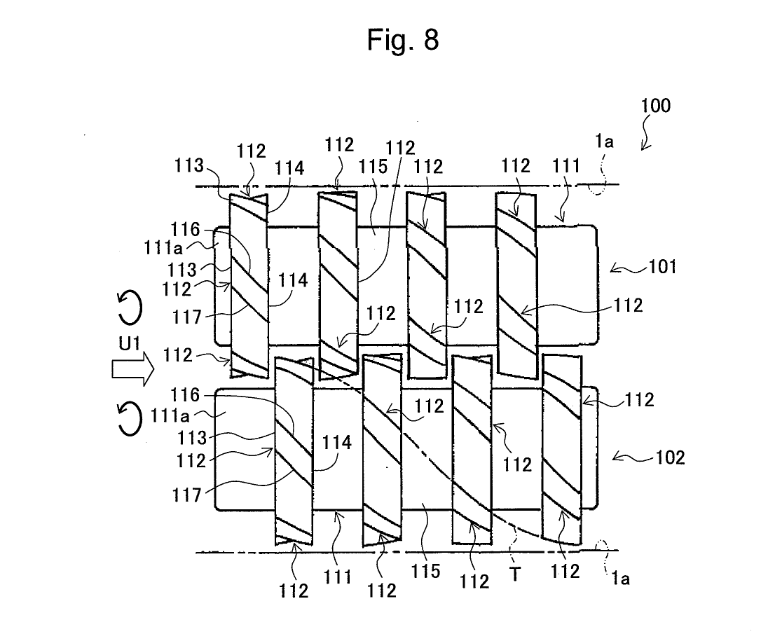

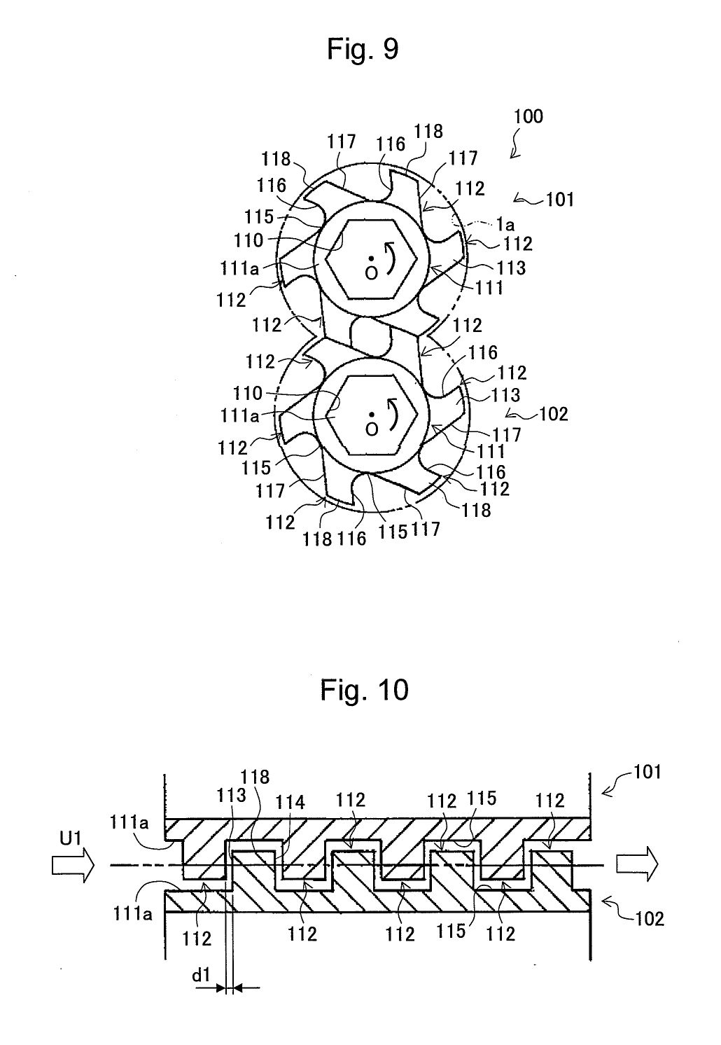

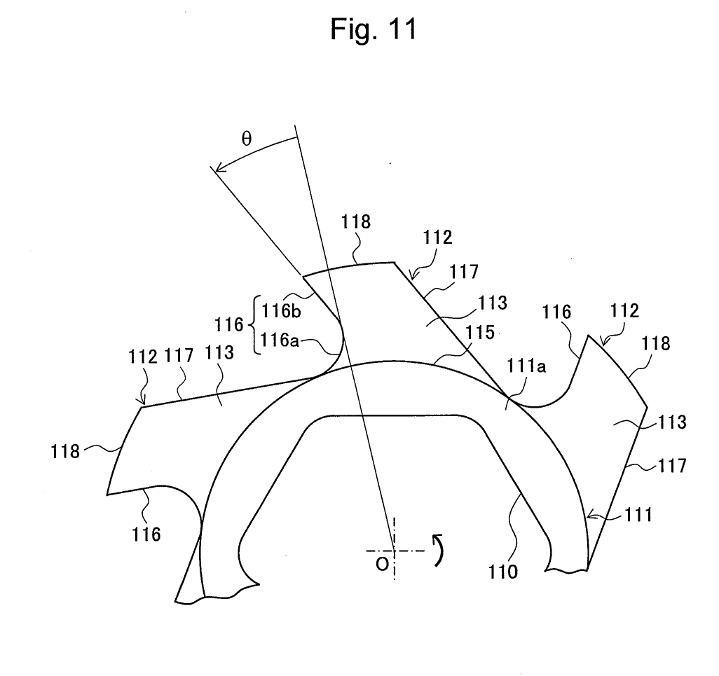

Description is now given of a configuration of the special gear kneader. FIG. 8 is a view showing a configuration of the special gear kneader, FIG. 9 is a view showing the special gear kneader shown in FIG. 8 from an arrow U1 direction that is a delivery direction of plant biomass feedstock, FIG. 10 is a schematic cross sectional view showing a gear fitting state of the special gear kneader of FIG. 8, and FIG. 11 is a partially enlarged view showing a tooth section shown in FIG. 9.

The special gear kneader 100, as shown in FIG. 8 or FIG. 9, is composed of a first rotor 101 and a second rotor 102. The first rotor 101 and the second rotor 102 are each structured to have a plurality of tooth sections 112 on a cylindrical shaft section 111.

As shown in FIG. 9, the shaft section 111 has a hexagonal through hole 110 formed along the central axis of the shaft section 111. The screw shaft 7 is inserted in the through hole 110 and fixed therein, and thereby the special gear kneader 100 can integrally rotate with the screw shaft 7.

As shown in FIG. 9, a plurality of the tooth sections 112 are protrudingly provided at predetermined intervals in a circumferential direction around the axis of the shaft section 111, and in this embodiment, the six tooth sections 112 are arranged at constant intervals. The number of the tooth sections 112 is not limited to the number in this embodiment, but may be one or more.

As shown in FIG. 8, a plurality of these tooth sections 112 are also provided at predetermined intervals in a delivery direction U1 that is an axial length direction of the shaft section 111, and in this embodiment, with the six tooth sections 112 consecutively provided in the circumferential direction around the axis being counted as one tooth section group, the tooth sections 112 are arranged to form total four tooth section groups in the delivery direction U1. The number of the tooth section groups is also not limited to the number in this embodiment, but may be two or more.

The tooth section 112 has a fixed thickness width along the axial length direction of the shaft section 111. A front surface 113 is formed along a radial direction of the shaft section 111 on the upstream side in the delivery direction, which is the front side in the axial length direction, while a rear surface 114 is formed along the radial direction of the first shaft section 111 on the downstream side in the delivery direction, which is the rear side in the axial length direction.

The tooth section 112 also includes, as shown in FIG. 9, tooth flanks 116, 117 which extend outward in a shaft diameter direction from a shaft barrel outer peripheral surface 115 of the shaft section 111 and which extend along the axial length direction, and a top surface 118 which continuously extend between top end portions of the tooth flanks 116 and 117.

As shown in FIG. 8, the tooth flanks 116, 117 are inclined so as to shift to the rear side in the rotation direction as they shift to the downstream side in the delivery direction, and they have a predetermined helical angle (lead). A spiral lead shown with an imaginary line T in FIG. 8 is obtained by connecting in the axial length direction the tooth flanks 116, 117 of a plurality of the tooth sections 112 which continue at predetermined intervals along the axial length direction. As the first rotor 101 or the second rotor 102 rotates in an arrow direction, the helical angle of the tooth flanks 116, 117 of the tooth section 112 ensures the performance to deliver the plant biomass feedstock in the arrow U1 direction.

As shown in FIG. 11, the tooth flank 116 out of a pair of the tooth flanks 116 and 117, which is positioned on the front side in the direction of rotation of the first rotor 101 or the second rotor 102, has a curved surface section 116a with a depressed circular cross section which smoothly rises from the shaft barrel outer peripheral surface 115 to the outside in the shaft diameter direction, and a flat-shaped vertical wall surface section 116b which continues to the curved surface section 116a and extends outward in the radial direction that is a direction away from the shaft section 111, and which is inclined to the front side in the rotation direction at an angle of gradient θ so as to shift to the front side in the rotation direction as it shifts outward in the radial direction.

On the contrary, the tooth flank 117 positioned on the rear side in the rotation direction has a flat shape which extends from the shaft barrel outer peripheral surface 115 to the outside in the radial direction and which is inclined so as to shift to the front side in the rotation direction as it shifts outward in the radial direction. In this embodiment, the tooth flank 117 is formed so as to be parallel to the vertical wall surface section 116b of the tooth flank 116.

The top surface 118 has an arc shape centering on axial center O of the shaft section 111, and is formed to face a round-shaped inner wall surface of the passage 1a with a predetermined gap between the top surface 118 and the inner wall surface as shown in FIG. 9.

As shown in FIG. 8, the first rotor 101 and the second rotor 102 are arranged in parallel such that between the tooth sections 112 arranged on the one shaft section 111 at predetermined intervals in the axial length direction, the tooth sections 112 of the other shaft section 111 are positioned, and so the tooth sections 112 of the first rotor 101 and the tooth sections 112 of the second rotor 102 are alternately positioned side by side in the axial length direction. Between the first rotor 101 and the second rotor 102, as shown in FIG. 10, a U-shaped clearance and a reversed U-shaped clearance are formed to continue in the arrow U1 direction that is the delivery direction, which ensures kneading performance and dispersion performance in the special gear kneader 100. A predetermined interval d1 is formed between the rear surface 114 of the tooth section 112 positioned on the upstream side in the delivery direction and the front surface 113 of the tooth section 112 which partially faces the rear surface 114 and which is positioned on the downstream side in the delivery direction.

Narrowing the interval d1 increases resistance in delivery of the plant biomass feedstock, and the narrowed interval can also be functioned as a resistance element for suppressing delivery of the plant biomass feedstock. Therefore, it is also preferable to arrange the gear kneader 100 in places where the high filling zone is formed in the hot compressed water treatment zone 12 in the cylinder 1.

Each of the shaft sections 111 of the first rotor 101 and the second rotor 102 has a boss section 111a protruding in the axial length direction more than the tooth section 112 positioned in the forefront on the upstream side in the delivery direction. The boss section 111a makes it possible to avoid collision of the plant biomass feedstock, which are delivered from the upstream side in the delivery direction with its flowing velocity maintained, with the front surface 113 of the tooth section 112 positioned in the forefront, to thereby prevent rapid compressive force and frictional force from being locally applied to the tooth section 112, and to decrease torque variation acting on a motor which rotationally drives the screw shaft.

Rotation timing of the first rotor 101 and the second rotor 102 is set such that as shown in FIG. 9 for example, the tooth section 112 of the one shaft section 111 and the tooth section 112 of the other shaft section 111 come near and intersect with each other at an intermediate position between the first rotor 101 and the second rotor 102.

According to the above-configured special gear kneader 100, the tooth flank 116 of the tooth section 112 formed on the front side in the rotation direction has the vertical wall surface section 116b which is inclined with an angle of gradient θ toward the front side in the rotation direction, and this makes it possible to reduce biasing force which is directed outward in the shaft diameter direction by rotation of the first rotation 101 and the second rotor 102 and which acts on the plant biomass feedstock. Therefore, it becomes possible to prevent the plant biomass feedstock from being moved outward by centrifugal force inside the passage 1a of the cylinder 1 and being locally subjected to compressive force and frictional force, and to thereby prevent occurrence of plugs (flocculated lumps).

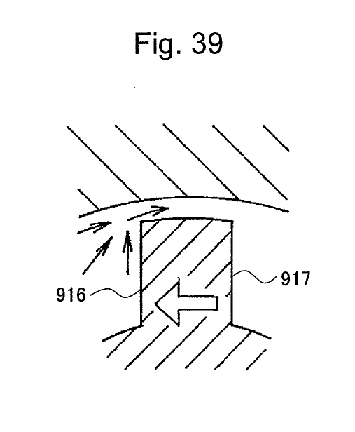

FIG. 38 is a schematic view of a gear kneader 910 included in a known twin screw extruder, and FIG. 39 is an enlarged view showing a principal part of FIG. 38. A tooth section 912 of the conventional gear kneader 910 is radically protruded from a shaft section 911 as shown in FIG. 38 and FIG. 39, and a tooth flank 916 out of a pair of tooth flanks 916 and 917, which is positioned on the front side in the rotation direction, has a flat shape which shifts to the rear side in the rotation direction as it shifts outward in the radial direction.

Therefore, nonliquid materials such as wood meals are blown by centrifugal force radially outwardly with respect to a first rotor 901 and a second rotor 902, and are locally subjected to compressive force and frictional force as shown with thin arrows in FIG. 39, as a result of which high-concentration and high-intensity plugs occur in an outermost part inside the passage 1a at an early stage. Due to compression resistance, frictional force and other properties of the plugs, rotation of the first rotor 901 and the second rotor 902 may be hindered, which leads to overload (motor overtorque) and difficulty in delivery.

Contrary to the conventional example, in the special gear kneader 100 of the present invention, the tooth flank 116 of the tooth section 112 positioned on the front side in the rotation direction has the vertical wall surface section 116b inclined with an angle of gradient θ toward the front side in the rotation direction as shown especially in FIG. 11, and thereby biasing force which is directed outward in the shaft diameter direction and which acts on the plant biomass feedstock can be reduced and occurrence of plugs in the passage 1a of the cylinder 1 can effectively be prevented. With the prevention of occurrence of plugs, it becomes possible to prevent the screw shaft 7 from deforming in the shaft diameter direction and to prevent in advance wear and overload caused by the tooth section 112 coming into contact with the passage 1a of the cylinder 1 from occurring.

In the case where the tooth sections 112, which are adjacent in the axial length direction, are moved in a direction of facing each other through rotation of the first rotor 101 and the second rotor 102 to shear the plant biomass feedstock, the plant biomass feedstock can be sheared with the vertical wall surface section 116b inclined with an angle of gradient θ toward the front side in the rotation direction, and thereby the force needed for shearing the plant biomass feedstock can be decreased. This makes it possible to decrease driving force of the extruder and can thereby achieve downsizing of the drive motor.

Moreover, since the tooth flank 116 of the tooth section 112 has a predetermined helical angle with respect to the axial length direction as shown with an imaginary line T in FIG. 8, the plant biomass feedstock can be biased to move from the upstream side to the downstream side in the delivery direction, the biasing force directed outward in the radial direction can be reduced and high compression in the outermost part inside the passage 1a of the cylinder 1 can be prevented.

Although the aforementioned special gear kneader 100 has been described by taking as an example the case where all of a plurality of the tooth sections 112 arranged at predetermined intervals in the axial length direction have a constant helical angle (lead), the degree of the helical angle may be changed corresponding to the positions that the tooth sections 112 are arranged in the axial length direction. For example, when the helical angle of the tooth flanks 116, 117 of the tooth section 112 positioned on the upstream side in the delivery direction is increased, and the helical angle of the tooth flanks 116, 117 of the tooth section 112 positioned on the downstream side in the delivery direction is decreased, a feed rate can be made larger on the downstream side than on the upstream side. The filling rate and concentration of the plant biomass feedstock can be changed corresponding to the positions of the tooth sections in the axial length direction, which allows more effective implementation of treatments such as shearing and diffusion.

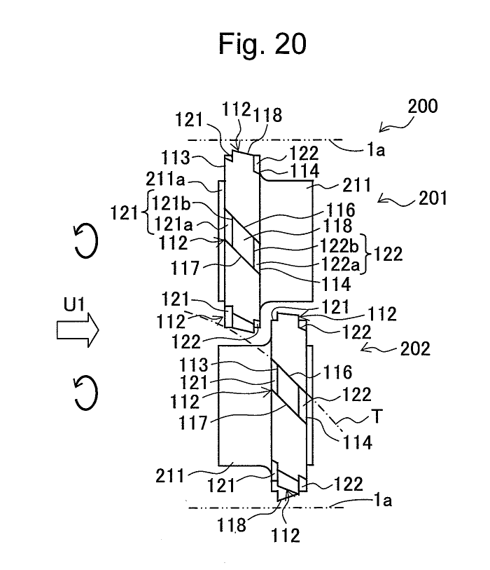

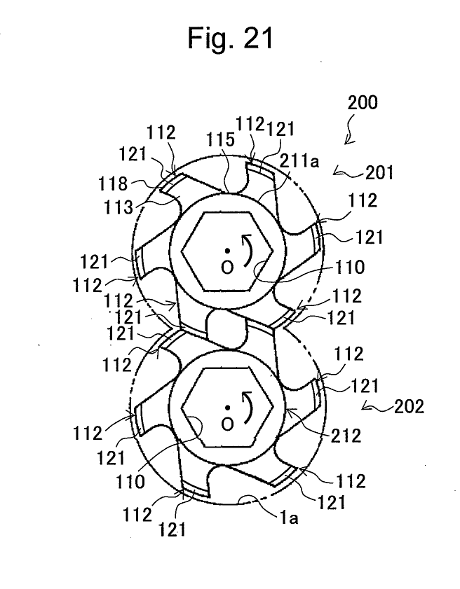

Next, an example of the special fluffer ring 200 having a characteristic configuration of the present invention is shown in FIG. 20 and FIG. 21. FIG. 20 is a view showing an example of a special fluffer ring, and FIG. 21 is a view of FIG. 20 viewed from an arrow U1 direction that is a delivery direction of plant biomass feedstock. It is to be noted that component members identical to those of the above-mentioned special gear kneader 100 are denoted by identical reference signs to omit detailed description.

The special fluffer ring 200 is composed of a first rotor 201 and a second rotor 202. The first rotor 201 and the second rotor 202 are each structured to have a plurality of the tooth sections 112 on a cylindrical shaft section 211. As shown in FIG. 21, a plurality of the tooth sections 112 are protrudingly provided at predetermined intervals in a circumferential direction around an axis of the shaft section 211. In this embodiment, the six tooth sections 112 are arranged at constant intervals.

As shown in FIG. 20, the first rotor 201 is structured to have the tooth sections 112 provided on the shaft section 211 at a position on the upstream side in the delivery direction that is the front side in the axial length direction, and to have the shaft section 211 protruding toward the downstream side in the delivery direction that is the rear side in the axial length direction. The second rotor 202 is structured to have the tooth sections 112 provided on the shaft section 211 at a position on the downstream side in the delivery direction, and to have the shaft section 211 protruding toward the upstream side in the delivery direction.

The first rotor 201 and the second rotor 202 are arranged such that the tooth sections 112 of the first rotor 201 face the shaft section 211 of the second rotor 202, while the tooth sections 112 of the second rotor 202 face the shaft section 211 of the first rotor 201, and the tooth sections 112 of the first rotor 201 and the tooth sections 112 of the second rotor 202 are arranged at positions closer to each other in the delivery direction.

A passage which bends in a crank form along the arrow U1 direction that is the delivery direction is formed between the first rotor 201 and the second rotor 202, which ensures kneading performance and dispersion performance in the special fluffer ring 200.

The first rotor 201 has a boss section 211a protruding in the axial length direction more than the tooth section 112. The second rotor 202 has the shaft section 211 provided on the upstream side of the tooth section 112 in the delivery direction.

The boss section 211a of the first rotor 201 and the shaft section 211 of the second rotor 202 make it possible to avoid collision of the plant biomass feedstock, which are delivered from the upstream side in the delivery direction with its flowing velocity maintained, with the front surface 113 of the tooth section 112 positioned in the forefront, to thereby prevent rapid compressive force from being locally applied to the tooth section 112, and to decrease torque variation acting on the motor which rotationally drives the screw shaft 7.

Rotation timing of the first rotor 201 and the second rotor 202 is set such that as shown in FIG. 21 for example, the tooth section 112 of the one shaft section 211 and the tooth section 112 of the other shaft section 211 come near and intersect with each other at an intermediate position between the first rotor 201 and the second rotor 202.

The tooth section 112 has stepped sections 121, 122 formed in a tip end part thereof. In the example shown in FIG. 20 and FIG. 21, the stepped section 121 is provided in all the six tooth sections 112 arranged in the circumferential direction around the axis in each of the first rotor 101 and the second rotor 102. It is not necessary to provide the stepped sections 121, 122 to all the tooth sections 112 included in the special fluffer ring 200. Settings of the tooth section 112 having the stepped sections 121, 122, such as arrangement positions, intervals and quantity are appropriately determined depending on the situation.

The stepped section 121 is formed on an edge part between the front surface 113 and the top surface 118 of the tooth section 112 along from the tooth flank 116 to the tooth flank 117, while the stepped section 122 is formed on an edge part between the rear surface 114 and the top surface 118 of the tooth section 112 along from the tooth flank 116 to the tooth flank 117. Therefore, the thickness width on a tooth tip side of each tooth section 112 is smaller than the thickness width on a tooth root side.

The stepped section 121, which is formed by notching the edge part between the front surface 113 and the top surface 118 of the tooth section 112 in a step shape, has an axial length-direction stepped surface 121a having a fixed width in the axial length direction at a position on the inside of the top surface 118 in the radial direction and a shaft diameter-direction stepped surface 121b having a fixed width in the shaft diameter direction at a position on the downstream side of the front surface 113 in the delivery direction.

The stepped section 122, which is formed by notching the edge part between the rear surface 114 and the top surface 118 of the tooth section 112 in a step shape, has an axial length-direction stepped surface 122a having a fixed width in the axial length direction at a position on the inside of the top surface 118 in the radial direction and a shaft diameter-direction stepped surface 122b having a fixed width in the shaft diameter direction at a position on the upstream side of the rear surface 114 in the delivery direction.

According to the above-configured special fluffer ring 200, the tooth section 112 has the vertical wall surface section 116b inclined with an angle of gradient θ toward the front side in the rotation direction, and this makes it possible to reduce biasing force which is directed outward in the shaft diameter direction and acts on the plant biomass feedstock. Therefore, it becomes possible to prevent high-concentration and high-intensity plugs (flocculated lumps) caused by compressive force and frictional force locally applied to the plant biomass feedstock in the passage 1a of the cylinder 1.

With the prevention of occurrence of plugs, it becomes possible to prevent the screw shaft 7 from deforming in the shaft diameter direction and to prevent in advance wear and overload caused by the tooth section 112 coming into contact with the passage 1a of the cylinder 1 from occurring.

In the case where the tooth sections 112, which are adjacent in the axial length direction, is moved in a direction of facing each other through rotation of the first rotor 201 and the second rotor 202 to shear the plant biomass feedstock, the plant biomass feedstock can be sheared with the vertical wall surface section 116b inclined with an angle of gradient θ toward the front side in the rotation direction, and the force needed for shearing the plant biomass feedstock can be decreased. This makes it possible to decrease driving force of the extruder and can thereby achieve downsizing of the drive unit.

Since the tooth flank 116 of the tooth section 112 has a helical angle shown with an imaginary line T, it becomes possible to deliver the plant biomass feedstock to the rear side in the shaft direction while preventing the plant biomass feedstock from being highly compressed toward the outside in the radial direction.

Since the tooth section 112 has the stepped sections 121, 122, the thickness width on the tooth tip side of the tooth section 112 is smaller than the thickness width on the tooth root side, and the tooth flank 116 is narrower on the tooth tip side of the tooth section 112 than on the tooth root side.

Therefore, it becomes possible to decrease feed components and shearing force in an outermost part inside the passage 1a where the plant biomass feedstock are high in concentration. This makes it possible to decrease torque for rotating the screw shaft 7 and to thereby achieve downsizing of the drive motor.

The stepped sections 121, 122 can alleviate compressive force and frictional force locally applied to the plant biomass feedstock by the tooth section 112, and can prevent the plant biomass feedstock from becoming highly concentrated and highly intensified in an outermost part inside the passage 1a at an early stage, and occurrence of plugs can be prevented.

The configuration of the special fluffer ring 200 is not limited to the above-mentioned embodiment, and various modifications and combinations are possible. For example, in the aforementioned embodiment, description has been made by taking as an example the case where the tooth section 112 of the special fluffer ring 200 has the two stepped sections 121 and 122, though the tooth section 112 can be structured to have either one of the stepped sections 121 and 122 or to have neither stepped sections 121 nor 122. It is also possible to structure the tooth section 112 provided with a chamfered section 131 (see description of a later-described special gear kneader 100 in an embodiment 3) for example.

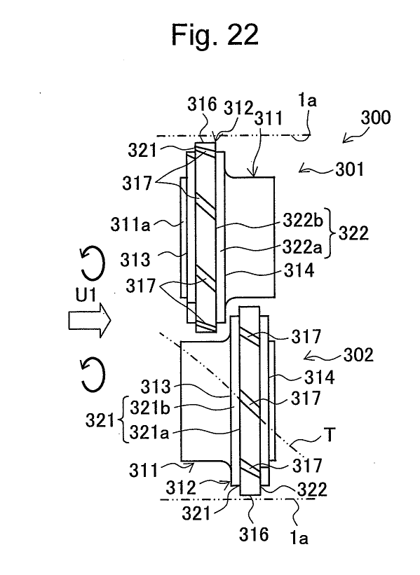

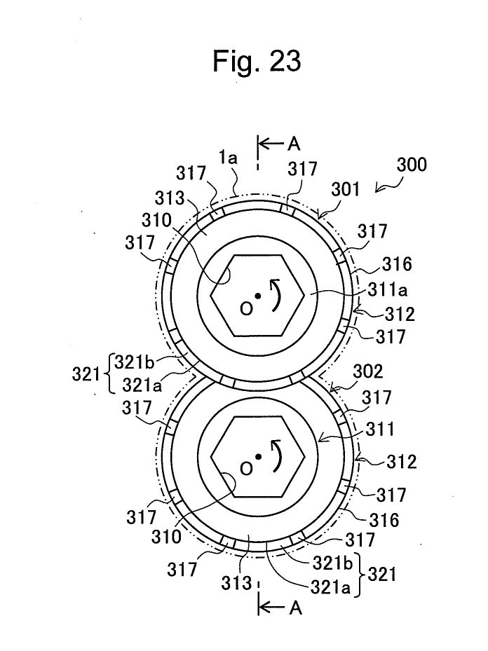

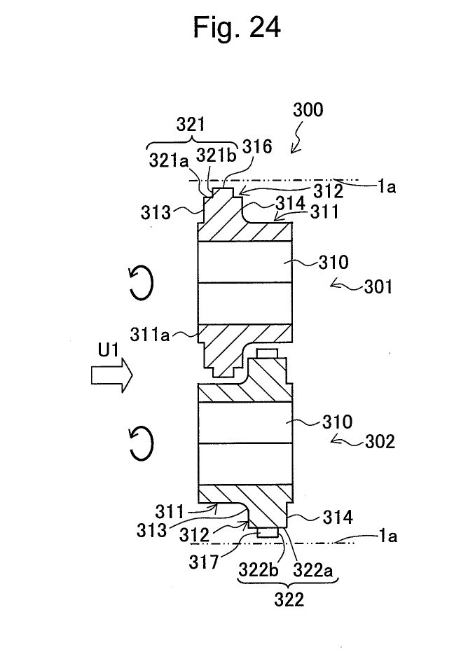

Next, an example of the special seal ring 300 having a characteristic configuration of the present invention is shown in FIG. 22 to FIG. 24. FIG. 22 is a view showing an example of a special seal ring, FIG. 23 is a view of FIG. 22 viewed from an arrow U1 direction that is a delivery direction of plant biomass feedstock, and FIG. 24 is a cross sectional view of FIG. 23 taken along line A-A.

The special seal ring 300, as shown in FIG. 22 to FIG. 24, is composed of a first rotor 301 and a second rotor 302. Each of the first rotor 301 and the second rotor 302 has a structure composed of a cylindrical shaft section 311 and an expanded section 312 expanded in one end portion of the shaft section 311.

As shown in FIG. 22, the first rotor 301 is structured to have the expanded section 312 provided on the shaft section 311 at a position on the upstream side in the delivery direction that is the front side in the axial length direction, and to have the shaft section 311 protruding toward the downstream side in the delivery direction that is the rear side in the axial length direction. The second rotor 302 is structured to have the expanded section 312 provided on the shaft section 311 at a position on the downstream side in the delivery direction, and to have the shaft section 311 protruding toward the upstream side in the delivery direction.

The first rotor 301 and the second rotor 302 are arranged such that the expanded section 312 of the first rotor 301 faces the shaft section 311 of the second rotor 302 and the expanded section 312 of the second rotor 302 faces the shaft section 311 of the first rotor 301, and the expanded section 312 of the first rotor 301 and the expanded section 312 of the second rotor 302 are arranged at positions closer to each other in the delivery direction.

As shown in FIG. 23, the first rotor 301 and the second rotor 302 are arranged such that the expanded sections 312 partially overlap with each other in the delivery direction at an intermediate position between the first rotor 301 and the second rotor 302, which ensures seal performance between the upstream side and the downstream in the delivery direction in the special seal ring 300.

The first rotor 301 has a boss section 311a protruding in the axial length direction more than the expanded section 312. The second rotor 302 has the shaft section 311 provided on the upstream side of the expanded section 312 in the delivery direction.

The boss section 311a of the first rotor 301 and the shaft section 311 of the second rotor 302 make it possible to avoid collision of the plant biomass feedstock, which are delivered from the upstream side in the delivery direction with its flowing velocity maintained, with a front surface 313 of the expanded section 312, and to thereby prevent rapid compressive force from being locally applied to the expanded section 312, and to decrease torque variation acting on the motor which rotationally drives the screw shaft 7.

As shown in FIG. 23, the shaft section 311 has a hexagonal through hole 310 formed along the central axis of the shaft section 311. The screw shaft 7 of an extruder is inserted in the through hole 310 and fixed therein, and thereby the special seal ring 300 can integrally rotate with the screw shaft.

The expanded section 312 is in a cylindrical short-shaft shape having a predetermined shaft-direction length which continues in an axial length direction of the shaft section 311 with a constant diameter. The size of the expanded section 312 is set such that an outer peripheral surface 316 of the expanded section 312 faces the inner wall surface of the passage 1a with a predetermined gap.

Lead grooves 317 are recessed on the outer peripheral surface 316 of the expanded section 312. As shown in FIG. 22, the lead groove 317 extends from the front surface 313 to a rear surface 134 of the expanded section 312 and communicates between the upstream side of the expanded section 31 in the delivery direction and the downstream side in the delivery direction.

The lead groove 317 has a predetermined helical angle (lead) so as to shift to the rear side in the rotation direction as it shifts to the downstream side in the delivery direction. In this embodiment, the lead groove 317 is formed so as to extend along a spiral imaginary line T shown in FIG. 22.

The lead groove 317 can pass the plant biomass feedstock which are delivered from the upstream side of the expanded section 312 in the delivery direction inside the passage 1a. Therefore, it becomes possible to prevent excessive high pressure on the upstream side of the special seal ring 300 in the delivery direction, and to thereby prevent occurrence of plugs on the upstream side in the delivery direction.

As the first rotor 301 and the second rotor 302 rotate in an arrow direction, the lead groove 317 can deliver plant biomass feedstock to the downstream side in the delivery direction with the helical angle of the lead groove 317. If the helical angle of the lead groove 317 is zero, i.e., if the lead groove 317 extends in parallel with the central axis of the shaft section 311, the capability of delivering plant biomass feedstock becomes zero, and the special seal ring 300 functions to shear and disassemble the plant biomass feedstock. At least one or more lead grooves 317 are provided, and in this embodiment, total eight lead grooves 317 are arranged at regular intervals in a circumferential direction as shown in FIG. 23.

The lead groove 317, which can generate turbulence in a flow of the plant biomass feedstock passing between the inner wall surface of the passage 1a and the special seal ring 300 and which can also impart feed components in a flow direction while relieving variation of the plant biomass feedstock positioned on the upstream side of the special seal ring 300, has a property of relieving pressure and fluidity, and enables the plant biomass feedstock to be kept in a smooth resistance and retention state.

As a result, it becomes possible to stabilize delivering resistance which suppresses delivery of the plant biomass feedstock in the passage 1a of the cylinder 1 and to retain pressure difference between the upstream side and the downstream side of the special seal ring 300. Therefore, it becomes possible to keep the pressure in the hot compressed water treatment zone 12 formed between, for example, the resistance element 31 and the resistance element 33 of the cylinder 1 and to suppress pressure variation in the hot compressed water zone so as to maintain the zone in a high-temperature and high-pressure state.

With the lead groove 317, delivery of plant biomass feedstock can be suppressed while some of the plant biomass feedstock can be guided to the downstream side of the cylinder 1. This makes it possible to prevent the pressure on the upstream side of the special seal ring 300 from becoming excessively high and to thereby prevent occurrence of plugs (flocculated lumps) on the upstream side of the special seal ring 300.

Stepped sections 321 and 322 are respectively provided on the expanded section 312 at a position on the upstream side in the delivery direction and at a position on the downstream side in the delivery direction. The stepped section 321 is formed to be peripherally continuous at an edge part between the front surface 313 and the outer peripheral surface 316, while the stepped section 322 is formed to be peripherally continuous at an edge part between a rear surface 314 and the outer peripheral surface 316.

The stepped section 321, which is formed by notching the edge part between the front surface 313 and the outer peripheral surface 316 of the expanded section 312 in a step shape, has an axial length-direction stepped surface 321a having a fixed width in the axial length direction at a position on the inside of the outer peripheral surface 316 in the radial direction and a shaft diameter-direction stepped surface 321b having a fixed width in the shaft diameter direction at a position on the downstream side of the front surface 313 in the delivery direction.

The stepped section 322, which is formed by notching the edge part between the rear surface 314 and the outer peripheral surface 316 of the expanded section 312 in a step shape, has an axial length-direction stepped surface 322a having a fixed width in the axial length direction at a position on the inside of the outer peripheral surface 316 in the radial direction and a shaft diameter-direction stepped surface 322b having a fixed width in the shaft diameter direction at a position on the upstream side of the rear surface 314 in the delivery direction.

The stepped section 321 can relieve compressive force and frictional force locally applied to the plant biomass feedstock by the expanded section 312, and can prevent the plant biomass feedstock from becoming highly concentrated and highly intensified in an outermost part located radially outwardly within the passage 1a at an early stage, and occurrence of plugs can be prevented.

The stepped section 321 can decrease a surface area of the front surface 313 of the expanded section 312. Therefore, compressive force and frictional force generated when the plant biomass feedstock, which were delivered from the upstream side in the delivery direction, come into contact with the front surface 313 of the expanded section 312 can be made relatively small. This makes it possible to decrease torque for rotating the screw shaft 7 and to thereby achieve downsizing of the drive motor.

It is to be understood that the configuration of the lead groove 317 is not limited to that in the aforementioned embodiment, and appropriately changing the number of lead grooves 317, size of the groove, and shape of the groove and the like makes it possible to easily change the relieving property and the filling rate.

### Embodiment 2

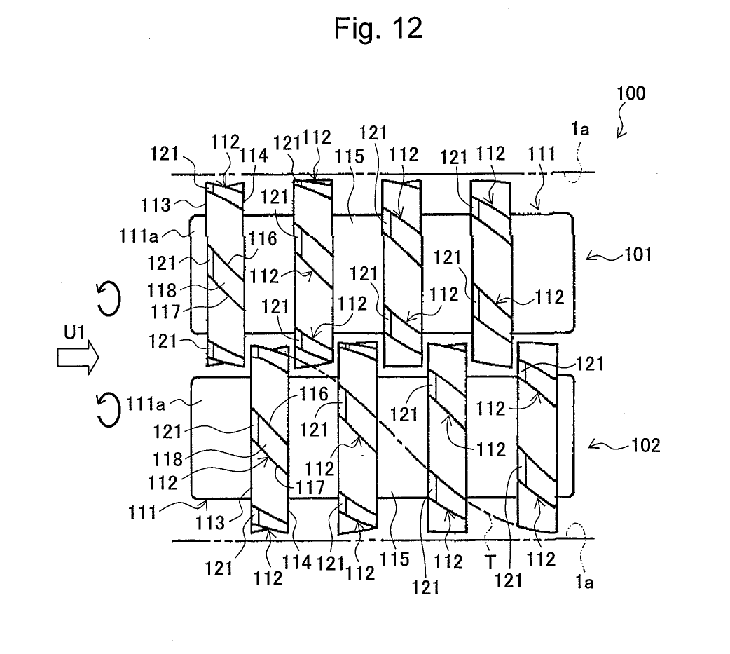

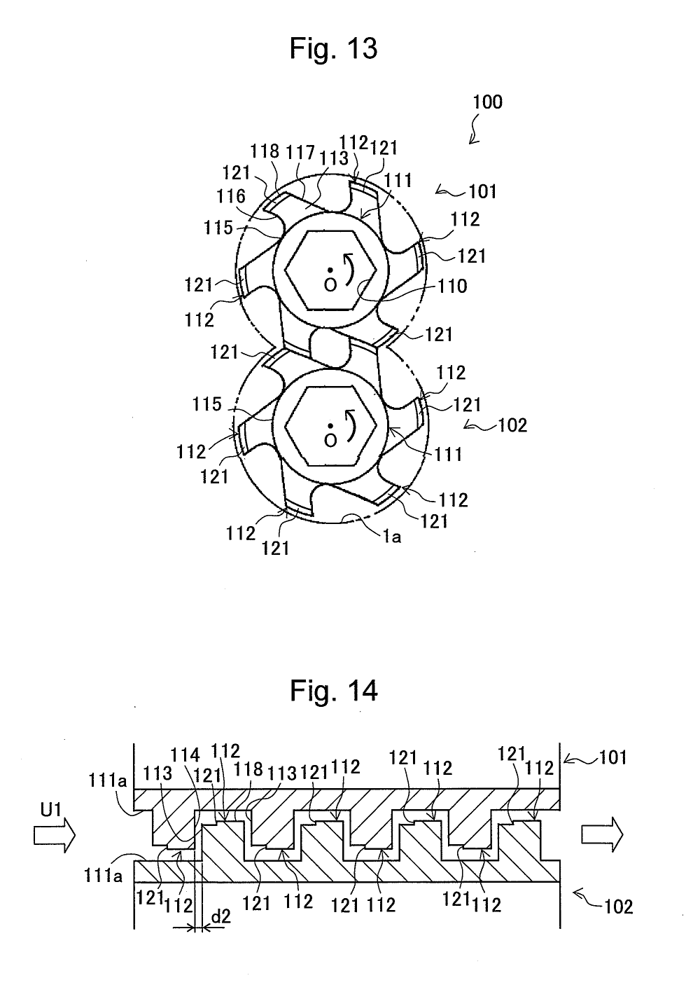

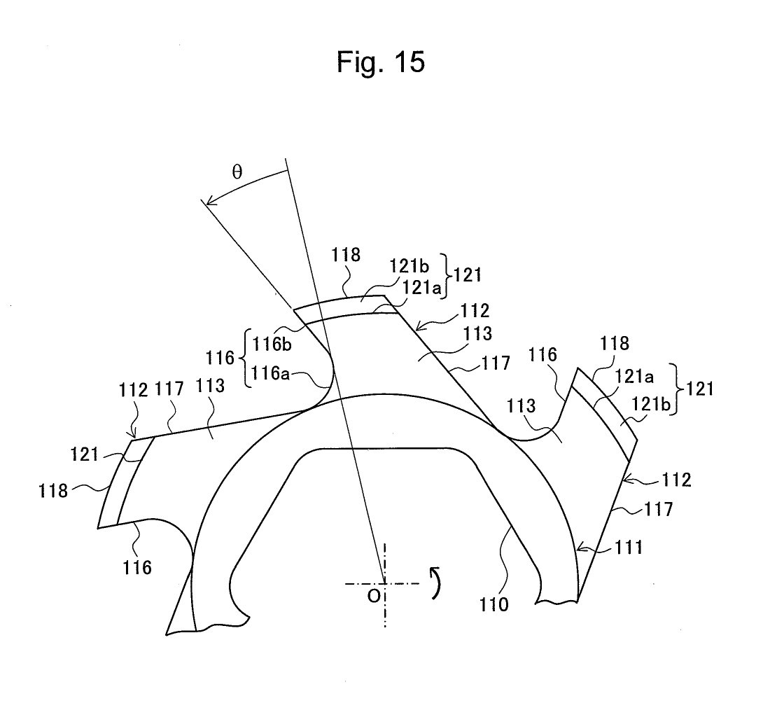

Description will be given of an embodiment 2 with reference to FIG. 12 to FIG. 15. In the embodiment 2, another example of a special gear kneader 100 will be described. FIG. 12 is a view showing another example of the special gear kneader, FIG. 13 is a view of the special gear kneader viewed from an arrow U1 direction shown in FIG. 12, FIG. 14 is a schematic view showing a gear fitting state of the special gear kneader, and FIG. 15 is a partially enlarged view showing a tooth section.

The special gear kneader 100 is configured to have a stepped section 121 formed at a tip end part of a tooth section 112 as shown in FIG. 12 and FIG. 13. In the example shown in FIG. 12 to FIG. 15, the stepped section 121 is provided in all the six tooth sections 112 arranged in a circumferential direction around an axis in each of a first rotor 101 and a second rotor 102.

It is not necessary to provide the stepped section 121 in all the tooth sections 112 included in the special gear kneader 100. Settings of the tooth section 112 having the stepped section 121, such as arrangement positions, intervals and quantity are appropriately determined depending on the situation.

As shown in FIG. 12 and FIG. 13 for example, the stepped section 121 is formed on an edge part between a front surface 113 and a top surface 118 of the tooth section 112 along from a tooth flank 116 to a tooth flank 117, and the thickness width of each tooth section 112 is set to be smaller on its tip end side than on its starting end side.

As shown in FIG. 14 and FIG. 15, the stepped section 121, which is formed by notching the edge part between the front surface 113 and the top surface 118 of the tooth section 112 in a step shape, has an axial length-direction stepped surface 121a having a fixed width in an axial length direction at a position on the inside of the top surface 118 in the radial direction and a shaft diameter-direction stepped surface 121b having a fixed width in the shaft diameter direction at a position on the downstream side of the front surface 113 in the delivery direction.

Since the tooth section 112 is formed such that the thickness width of the tooth section 112 is smaller on a tip end side than on a starting end side with the stepped section 121, it becomes possible to decrease feed components and shearing force in the outside in the radial direction within a passage 1a where the plant biomass feedstock are high in density. This makes it possible to decrease torque for rotating the screw shaft and to thereby achieve downsizing of the drive motor.

The stepped section 121 can relieve compressive force and frictional force locally applied to the plant biomass feedstock by the tooth section 112, and can prevent the plant biomass feedstock from becoming highly densified and highly intensified in an outermost part positioned in the outside in the radial direction within the passage 1a at an early stage, and occurrence of plugs can be prevented.

### Embodiment 3

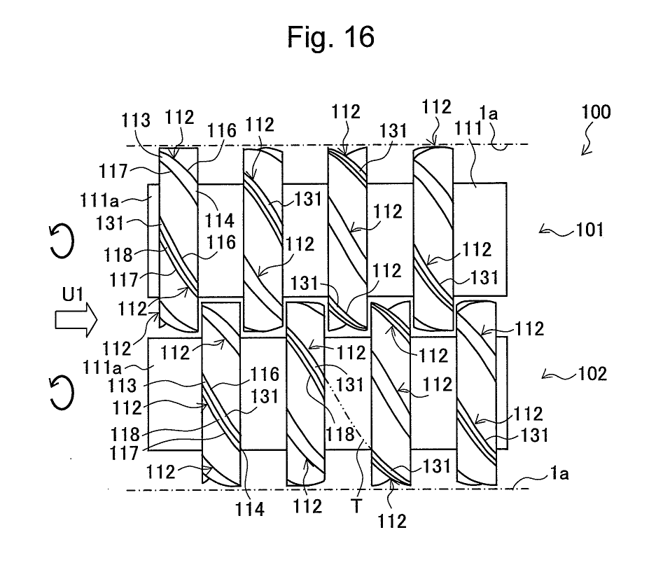

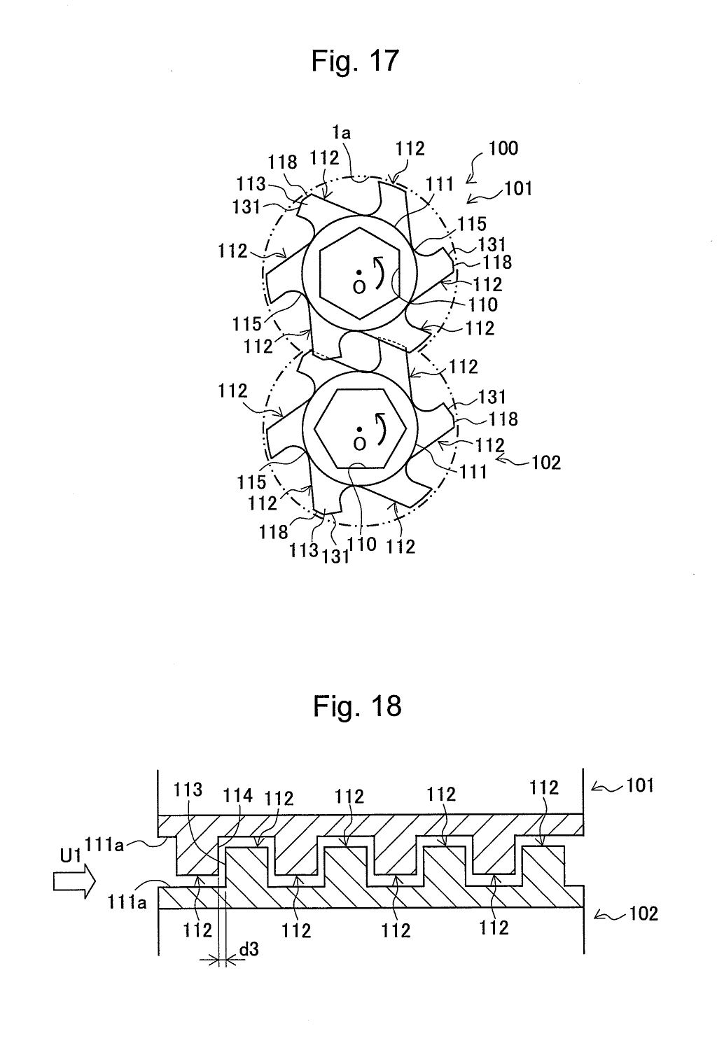

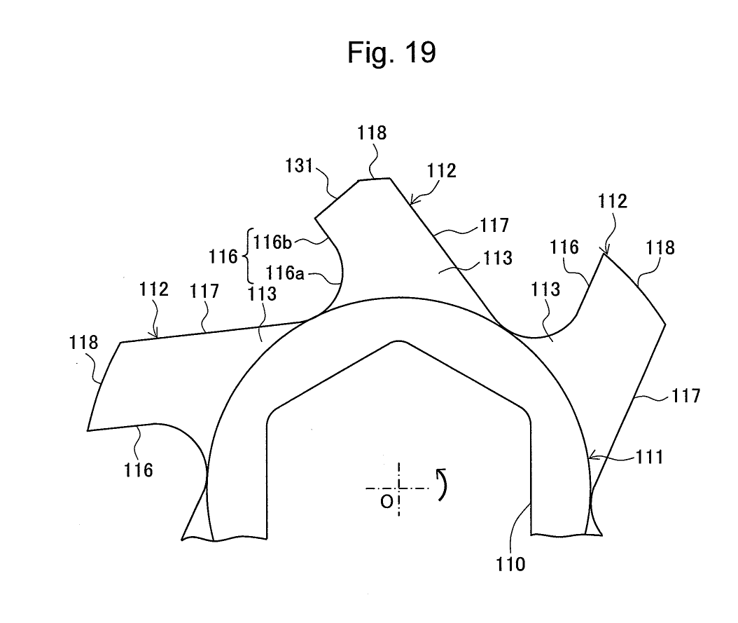

Description will be given of an embodiment 3 with reference to FIG. 16 to FIG. 19. In the embodiment 3, still another example of a special gear kneader 100 will be described. FIG. 16 is a view showing another example of the special gear kneader, FIG. 17 is a view of the special gear kneader viewed from an arrow U1 direction shown in FIG. 16, FIG. 18 is a schematic view showing a gear fitting state of the special gear kneader, and FIG. 19 is a partially enlarged view showing a tooth section.

The special gear kneader 100 is configured to have a chamfered section 131 at a tip end part of a tooth section 112 as shown especially in FIG. 17 and FIG. 19. The chamfered section 131 needs not be provided in all the tooth sections 112 included in the special gear kneader 100, but may be provided in at least one of a plurality of the tooth sections 112 arranged at predetermined intervals in the circumferential direction around the axis or may be provided in at least one of a plurality of the tooth sections 112 arranged at predetermined intervals in the axial length direction.

Settings of the tooth section 112 having the chamfered section 131, such as arrangement positions, intervals and quantity are appropriately determined depending on the situation. In the example shown in FIG. 16 to FIG. 19, the chamfered section 131 is provided in the three tooth sections 112 out of the six tooth sections 112 arranged in the circumferential direction around the axis in each of a first rotor 101 and a second rotor 102, the tooth section 112 with the chamfered section 131 and the tooth section 112 without the chamfered section 131 being alternately arranged side by side in the circumferential direction around the axis.

As shown in FIG. 16 and FIG. 19, the chamfered section 131 is formed on an edge part between a tooth flank 116 and a top surface 118 along from a front surface 113 to a rear surface 114 of the tooth section 112, and has a flat shape inclined so as to shift outward in the shaft diameter direction as it shifts to the rear side in the rotation direction.

The chamfered section 131 is provided in a tip end part of the tooth section 112, and has an inclination so as to shift outward in the shaft diameter direction as it shifts to the rear side in the rotation direction, and thereby some of the plant biomass feedstock, which are present on the front side of the tooth section 112 in the rotation direction, can be passed through a space between the chamfered section 131 and the inner wall surface of a passage 1a and can be moved to the rear side of the tooth section 112 in the rotation direction.

Moreover, the chamfered section 131 makes it possible to decrease feed components and shearing force in the outside in the radial direction within the passage 1a where the plant biomass feedstock are high in density. This makes it possible to decrease torque for rotating a screw shaft 7 and to thereby achieve downsizing of the drive motor.

The chamfered section 131 can also relieve compressive force and frictional force locally applied to the plant biomass feedstock by the tooth section 112, and can prevent the plant biomass feedstock from becoming highly densified and highly intensified in an outermost part positioned in the outside in the radial direction within the passage 1a at an early stage, and occurrence of plugs can be prevented.

Between the first rotor 101 and the second rotor 102, as shown in FIG. 18, a U-shaped clearance and a reversed U-shaped clearance are formed to be continuous in the arrow U1 direction that is the delivery direction. The chamfered section 131 can prevent high density and high intensity of the plant biomass feedstock which are present on the front side of the tooth section 112 in the rotation direction. Therefore, an interval d3 between the rear surface 114 of the tooth section 112 positioned on the upstream side in the delivery direction and the front surface 113 of the tooth section 112 which partially faces the rear surface 114 and is positioned on the downstream side in the delivery direction can be made smaller (d3<d1, d3<d2). As a result, the plant biomass feedstock can be further micronized in between a plurality of the tooth sections 112 arranged along the axial length direction.

The configuration of the special gear kneader 100 is not limited to those in each of the above-mentioned embodiments, and various combinations are possible. For example, the special gear kneader 100 may be configured to have both the tooth section 112 having a stepped section 121 and the tooth section 112 having the chamfered section 131, and the tooth section 112 may also be configured to have both the stepped section 121 and the chamfered section 131.

### Embodiment 4

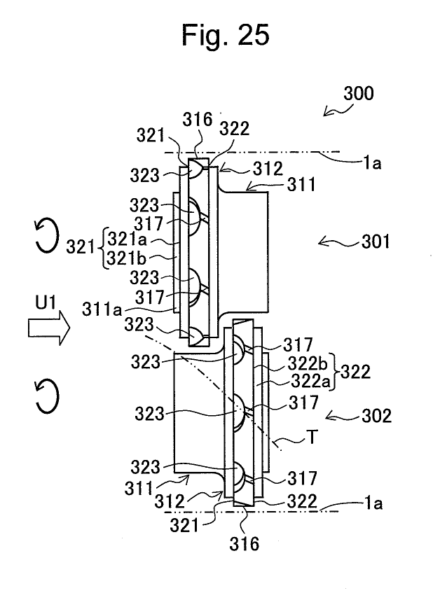

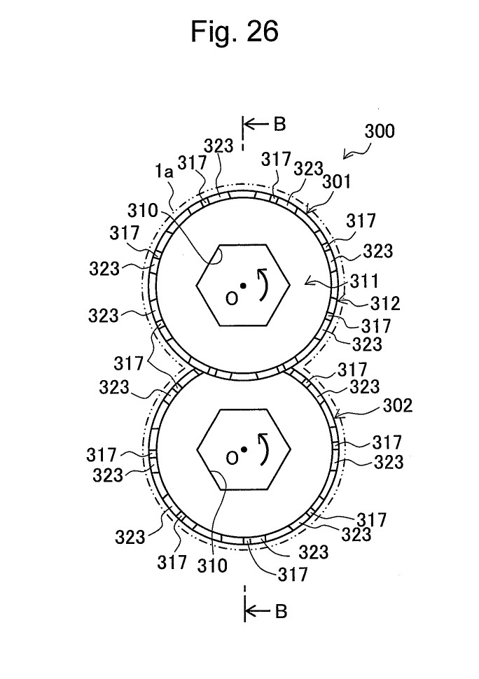

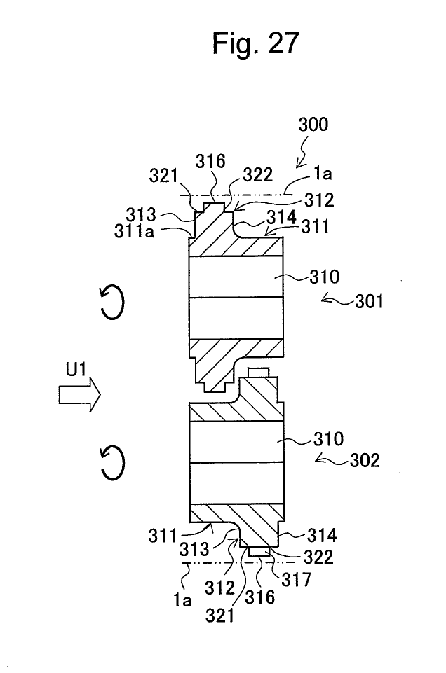

Description will be given of an embodiment 4 with reference to FIG. 25 to FIG. 27. In the embodiment 4, another example of a special seal ring 300 will be described. FIG. 25 is a view showing an example of the special seal ring, FIG. 26 is a view of FIG. 25 viewed from an arrow U1 direction that is a delivery direction of plant biomass feedstock, and FIG. 27 is a cross sectional view of FIG. 25 taken along line B-B.

The special seal ring 300 is structured to have a recess section 323 on an outer peripheral surface 316 as shown in FIG. 25 and FIG. 26. In the recess section 323, an upstream side in the delivery direction is opened toward the front side, and a downstream side in the delivery direction is narrower than the upstream side in the delivery direction and is in a shape communicating with an upstream section of a lead groove 317.

Total eight lead grooves 317 are provided on the outer peripheral surface 316 of an expanded section 312. The recess section 323 is respectively provided at positions corresponding to each of these lead grooves 317.

As shown in FIG. 26 for example, the recess section 323 has a depth substantially equal to a groove depth of the lead groove 317. As shown in FIG. 25 for example, the recess section 323 has a semicircular shape which protrudes toward the downstream side in the delivery direction from a shaft diameter-direction stepped surface 321b of a stepped section 321. An end portion of the recess section 323 on the downstream side in the delivery direction is connected to the lead groove 317.

The recess section 323 can agitate a part of plant biomass feedstock while moving the plant biomass feedstock to an outermost part within a passage 1a. Therefore, it becomes possible to make the flow of the plant biomass feedstock between the special seal ring 300 and the passage 1a more complicated, to seal a space between the upstream side and the downstream side of the special seal ring 300, and to keep the pressure in a zone(s) formed between a seal ring 330 provided upstream of the passage 1a and the special seal ring 300 provided downstream.

Since the recess section 323 has a semicircular shape which becomes narrower toward the downstream side in the delivery direction, it becomes possible to relieve compressive force and frictional force locally applied to the plant biomass feedstock by the outer peripheral surface 316 of the special seal ring 300, and to prevent the plant biomass feedstock from becoming highly densified and highly intensified in the outermost part at an early stage, and occurrence of plugs can be prevented.

It is to be noted that the shape of the recess section 323 is not limited to the semicircular shape, and any shape including irregular shapes, such as semielliptical shape and triangle shape can be used as long as the flow of the plant biomass feedstock can be complicated.

### Embodiment 5

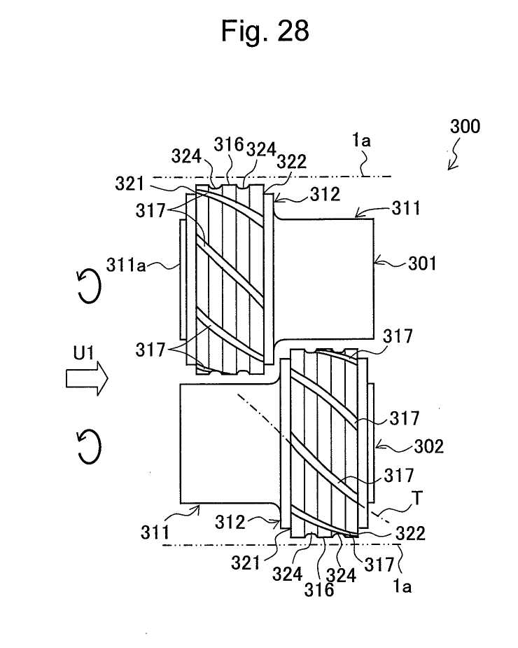

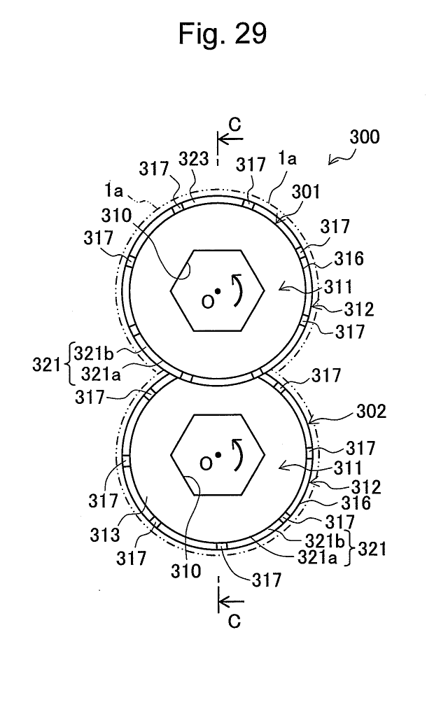

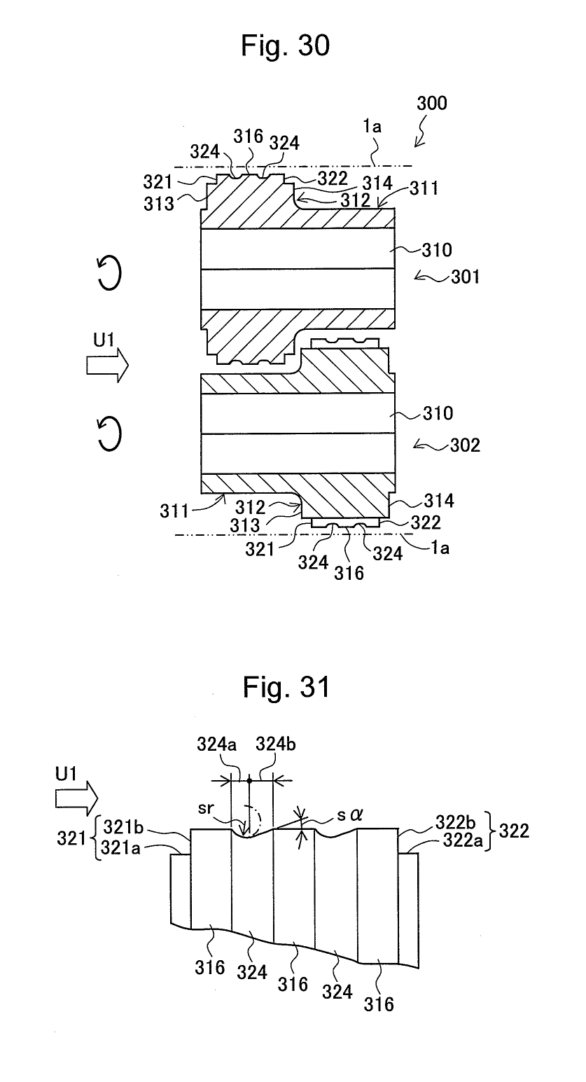

Next, a still another example of a special seal ring 300 is shown in FIG. 28 to FIG. 31. FIG. 28 is a view showing an example of the seal ring, FIG. 29 is a view of FIG. 28 viewed from an arrow U1 direction that is a delivery direction of plant biomass feedstock, FIG. 30 is a cross sectional view of FIG. 29 taken along line C-C, and FIG. 31 is an enlarged view showing a principal part of FIG. 28.

The special seal ring 300 is structured to have at least one or more circumferential grooves 324 recessed in an outer peripheral surface 316 of an expanded section 312. As shown in FIG. 28, the circumferential groove 324 is formed so as to extend along the circumferential direction of the outer peripheral surface 316, and the two circumferential grooves are provided at a predetermined interval in the axial length direction in this embodiment. As shown in FIG. 31, the circumferential groove 324 includes a depressed curve section 324a forming a portion of the circumferential groove 324 on the upstream side in the delivery direction and a tapered section 324b forming a portion of the circumferential groove 324 on the downstream side in the delivery direction.

The depressed curve section 324a is formed to have a depressed circular arc-shaped cross section with a constant radius of curvature sr. The tapered section 324b is formed to have an inclined cross section which has an angle of gradient sa and which gradually shifts outward in the radial direction as it shifts toward the downstream side in the delivery direction from the depressed curve section 324a.

Therefore, when the plant biomass feedstock which were delivered from the upstream to the downstream in a passage 1a moves from a position facing the outer peripheral surface 316 to a position facing the circumferential groove 324, the depressed curve section 324a of the circumferential groove 324 can rapidly lower the pressure acting on the plant biomass feedstock and can relieve variation in pressure and flow. The tapered section 324b of the circumferential groove 324 can gradually increase the variation in pressure and flow which act on the plant biomass feedstock.

This relief and increase in pressure and the like of the plant biomass feedstock are repeated with a plurality of the circumferential grooves 324, and thereby pressure and resistance applied to a flow direction of the plant biomass feedstock can be smoothed and safer seal resistance (fluidity) can be obtained. This sealing performance is particularly effective in a high-temperature and high-pressure zone where the plant biomass feedstock are highly densified at high speed.

The number of the circumferential grooves 324 may be one, and may also be three or more. The circumferential groove 324 may be configured to have a slight helical angle so as to gradually shift to the downstream side in the delivery direction as it shifts to the rear side in the rotation direction, such that the variation in pressure which acts on the plant biomass feedstock is relieved.

### Embodiments 6 to 8

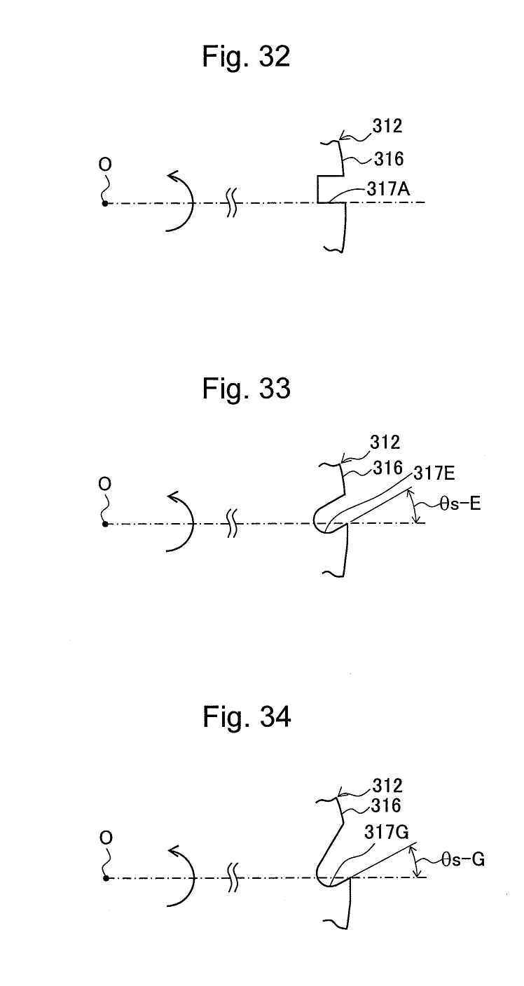

FIG. 32 to FIG. 34 are views showing a lead groove provided in a seal ring in cross section.

A lead groove 317 is provided on an outer peripheral surface 316 of an expanded section 312. The lead groove 317 extends from a front surface 313 to a rear surface 134 of the expanded section 312 and communicates between the upstream side in the expanded section 312 in the delivery direction and the downstream side in the delivery direction.

A lead groove 317A in an embodiment 6 shown in FIG. 32 has generally a U-shaped groove shape in cross section formed by notching the outer peripheral surface 316 along a radial direction. A lead groove 317E in an embodiment 7 shown in FIG. 33 has generally a U-shaped groove shape in cross section formed by notching the outer peripheral surface 316 toward the rear side in the rotation direction so as to have a predetermined angle of θs-E with respect to the radial direction. A lead groove 317G in an embodiment 8 shown in FIG. 34 has generally a V-shaped groove shape in cross section formed by notching the outer peripheral surface 316 toward the rear side in the rotation direction so as to have a predetermined angle of θs-G with respect to the radial direction.

Feeding force generated through agitation and flow with the lead grooves 317A, 317E, 317G is larger in order of the lead grooves 317A, 317E, 317G (317A<317E<317G), and the relieving property can arbitrarily be set with groove conditions and size, and therefore flow resistance of the plant biomass feedstock can be changed corresponding to the external diameter of the expanded section 312.

The above-described screw segments are not necessarily all be used at the same time, but are suitably selected depending on conditions and the like and are used being attached to the screw shaft 7.

Although each embodiment of the present invention has been described in full detail with reference to drawings, it should be understood that specific configurations are not limited to the embodiments described, and various modifications in design which come within the scope and the spirit of the present invention are therefore intended to be embraced therein.

For example, screw lines arranged in the passage 1a of the cylinder 1, helical angles, pitches, a length/diameter ratio, the number of screws and paddles and the like may suitably be selected where necessary. Although description has been made by taking the case of a twin screw extruder as an example, the present invention is not limited thereto and is applicable to single screw extruders or extruders with triple screws or more.

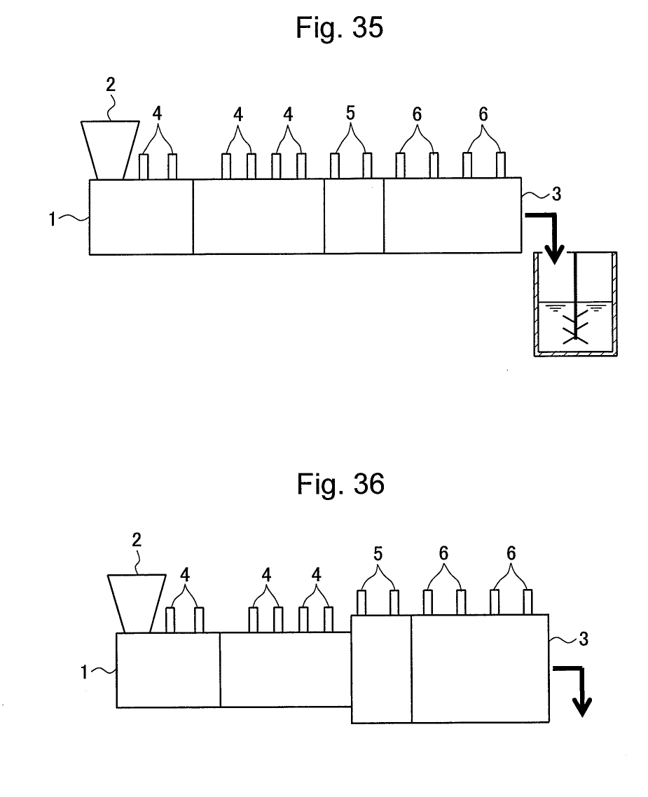

FIG. 35 is a schematic view showing another embodiment of a twin screw extruder in this embodiment. As shown in FIG. 35, the screw extruder may include a plurality of decomposing agent feed parts 4, coolant feed parts 5, and enzyme feed parts 6 along a flow direction of the cylinder 1. According to this configuration, decomposing agents, coolants, and enzymes may be fed at optimal timing in response to treatment states of the plant biomass feedstock in the passage 1a.

The screw extruder may have a configuration in which the diameter of the cylinder 1 is expanded in a halfway position as shown in FIG. 36. According to this configuration, a flow rate in the passage 1a can be decreased in the large diameter section on the downstream side, and a longer time can be ensured for such steps as the cooling step and the saccharification preparation step.

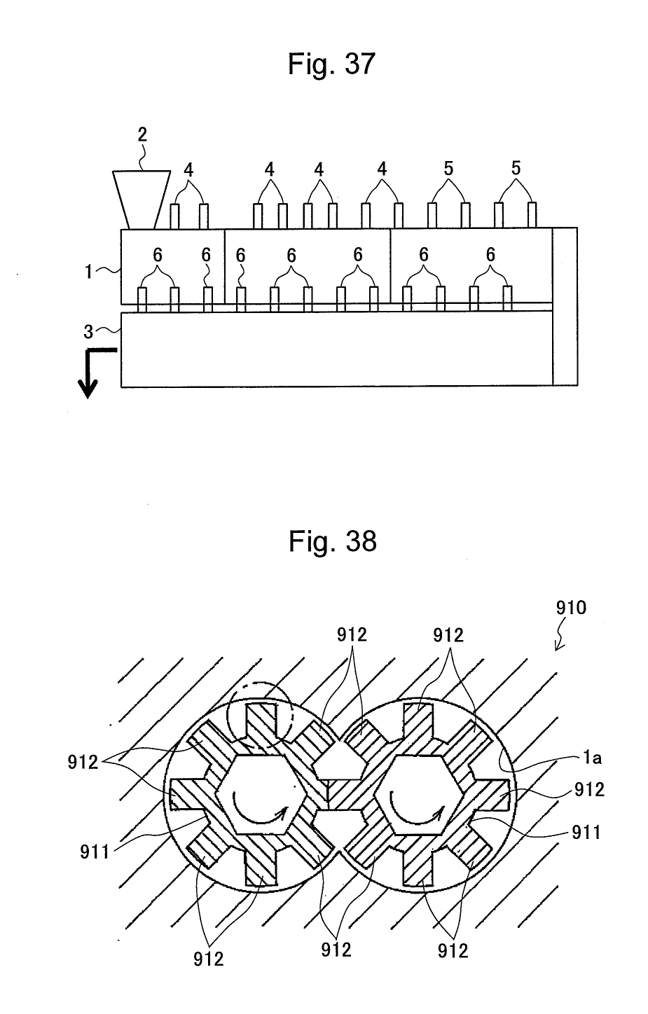

The screw extruder may also be structured to make a U-turn in a halfway position in the cylinder 1 as shown in FIG. 37. According to this structure, a longer length can be provided for the cylinder 1, and therefore the saccharification and fermentation treatments, which are subsequent to the treatment in the saccharification preparation zone 14, may also be performed in the cylinder 1.

1. A plant biomass pretreatment method for performing pretreatment to produce ethanol from the plant biomass with use of an enzyme, the method comprising continuously performing in sequence, inside an extruder, pretreatment steps of:
coarsely crushing the plant biomass to a predefined size or smaller;
adding a decomposing agent to the coarsely crushed plant biomass;
applying a hot compressed water treatment to the plant biomass with the decomposing agent added thereto; and
performing saccharification preparation for mixing the plant biomass with the hot compressed water treatment applied thereto with an enzyme for saccharifying the plant biomass.

2. The plant biomass pretreatment method according to claim 1, wherein
the extruder comprises:
a cylinder having a passage which includes a feed port formed for feeding the plant biomass in one end and a discharge port formed for discharging a material to be pretreated in the other end; and
a screw line which is arranged inside the passage of the cylinder and which includes a delivery section for delivering the plant biomass toward the discharge port, a kneading section for kneading the plant biomass, and a resistance element for providing delivering resistance to the plant biomass, the extruder having in sequence from an upstream side to a downstream side in the passage of the cylinder:
a coarse crushing zone for coarsely crushing the plant biomass to a predefined size or smaller;
a hot compressed water treatment zone for applying a hot compressed water treatment to the plant biomass coarsely crushed in the coarse crushing zone;
a cooling zone for cooling the plant biomass with the hot compressed water treatment applied thereto in the hot compressed water treatment zone;
a saccharification preparation zone for mixing the plant biomass cooled in the cooling zone with an enzyme; and
a discharge zone for discharging the plant biomass mixed with the enzyme in the saccharification preparation zone as a pretreated material.

3. The plant biomass pretreatment method according to claim 2, wherein a screw line having at least one or more types of screw segments, including a gear kneader or a fluffer ring, is placed in a plant biomass high filling zone formed by the resistance element of the screw line on an upstream side of the resistance element.

4. The plant biomass pretreatment method according to claim 2, wherein a screw line having at least one or more types of screw segments, including a forward kneading disk, a backward kneading disk, an perpendicular kneading disk, a gear kneader, and a fluffer ring, is placed in the coarse crushing zone.

5. The plant biomass pretreatment method according to claim 2, wherein a screw line having at least one or more types of screw segments, including a reverse full flight, a gear kneader, or a fluffer ring, is placed in the hot compressed water treatment zone, a resistance element having a seal ring is provided respectively in an upstream end and in a downstream end of the hot compressed water treatment zone, and the plant biomass is sheared and kneaded under pressure and heat in the hot compressed water treatment.

6. The plant biomass pretreatment method according to claim 5, wherein the resistance elements placed in the hot compressed water treatment zone are set such that the resistance element on a downstream side is higher in resistance than the resistance element on an upstream side.

7. The plant biomass pretreatment method according to claim 2, wherein a decomposing agent feed part for feeding a decomposing agent to the hot compressed water treatment zone in the passage, a coolant feed part for feeding a coolant to the cooling zone, and an enzyme feed part for feeding an enzyme to the saccharification preparation zone are each provided.

8. The plant biomass pretreatment method according to claim 2, wherein a plurality of the decomposing agent feed parts are provided at predetermined intervals along the passage of the cylinder, and a feed amount of the decomposing agent is set to be higher on the upstream side than on the downstream side.

9. The plant biomass pretreatment method according to claim 2, wherein the feed amount of the decomposing agent is set at 5 to 150 weight parts with respect to 100 weight parts of the plant biomass.

10. The plant biomass pretreatment method according to claim 2, wherein pressure and heat are applied to the extruder with a pressure inside the cylinder being 1 to 30 MPa and a temperature of the hot compressed water treatment zone being 130° C. to 350° C.

11. The plant biomass pretreatment method according to, claim 2 wherein a screw line having at least one or more types of screw segments, including a forward kneading disk, a backward kneading disk, and an perpendicular kneading disk, is placed in the discharge zone.

12. The plant biomass pretreatment method according to claim 2, wherein the cylinder comprises a vent in the discharge zone for discharging gas inside the passage, and the gas inside the cylinder is discharged through the vent.
